(19) United States
(12) Patent Application Publication
(10) Pub. No.: US 2013/0040898 A1
(43) Pub. Date: Feb. 14, 2013

(54) METHODS OF TREATMENT OF PATIENTS AT INCREASED RISK OF DEVELOPMENT OF ISCHEMIC EVENTS AND COMPOUNDS HEREOF

(71) Applicant: Pär Johansson, Dosjebro (SE)

(72) Inventor: Pär Johansson, Dosjebro (SE)

(21) Appl. No.: 13/643,366

(22) PCT Filed: Apr. 29, 2011
(86) PCT No.: PCT/DK2011/050142

§ 371 Date: Oct. 25, 2012

(30) Foreign Application Priority Data
Apr. 29, 2010 (DK) PA 2010 70181

(51) Int. Cl. A61K 38/12; A61P 9/10

(52) U.S. Cl. 514/21.1

(57) ABSTRACT

The present invention relates to compounds for treatment that protects the endothelium, prevents pathologic thrombus formation in the microcirculation and preserves platelet number and function and thus may be related to treatment or prevention of ischemic events in patients with cardiovascular disease. The present invention is particularly useful for patients having or being at increased risk of development of an ischemic event such as an acute myocardial infarction and/or no-reflow phenomena and/or ischemia-reperfusion injury by administration of agent(s) modulating and/or preserving endothelial integrity. The compounds may be administered in combination with standard treatment of acute cardiovascular ischemic events such as Platelet inhibitors such as aspirin (ASA), Thienopyridins, GPIIb/IIIa inhibitors), Parenteral anticoagulants such as unfractioned heparin (UFH), bivalirudin, enoxaparin, and fondaparinux, Verapamil, Adenosine, Sodium nitroprusside, Nitroglycerin, Epinephrine, Beta-blockers and surgical methods such as percutaneous coronary intervention (PCI), PCI with thrombus aspiration, PCI with stents.

All patent and non-patent references cited in the application, or in the present application, are also hereby incorporated by reference in their entirety.

### FIELD OF THE INVENTION

The present invention relates to a novel use of compounds that protect the endothelium, prevent pathologic thrombus formation in the microcirculation and/or preserve platelet number and function in the circulation and thus may be related to minimizing or preventing development of cardiovascular ischemic events, and, hence, death in patients with cardiovascular disease by administration of agent(s) that limit the platelets ability to aggregate and form clots and modulate/preserve endothelial integrity.

### BACKGROUND OF THE INVENTION

Under physiologic conditions, platelet aggregation and haemostasis is prevented by the vascular endothelium. The endothelium provides a physical barrier and secretes platelet inhibitory products, such as prostacycline (PGI2) and nitric oxide (NO). These compounds regulate the adhesiveness of platelets and the activation state of the platelet receptor GPIIb/IIIa in a paracrine way and also maintain the endothelium in a quiescent state through autocrine mechanisms [Zardi et al 2005].

With endothelial activation or injury (trauma, critical illness like sepsis, atherosclerosis, any disease accompanied by inflammation), platelets adhere to the endothelium or subendothelium, respectively.

Atherosclerotic lesions (atheromata) are asymmetric focal thickenings of the innermost layer of the artery, the intima. Myocardial infarction occurs when the atheromatous process prevents blood flow through the coronary artery. Activation of plaque rather than stenosis precipitates ischemia and infarction. Coronary spasm and/or endothelial dysfunction may be involved to some extent (cardiac syndrome X, angina X), but most cases of infarction are due to the formation of an occluding thrombus on the surface of the plaque as a result of coronary thrombosis: plaque rupture and endothelial erosion enabling platelets adhere to the endothelium or subendothelium, respectively.

This adhesion activates platelets, causes a shape change and a release reaction where ADP and other potent endogenous platelet agonists are released. The platelet membrane integrin receptor, GPIIb/IIIa, becomes activated. Fibrinogen binds to this receptor, effectively cross-linking platelets to form a platelet plug.

It has become increasingly apparent that clinical manifestations of myocardial ischemia are associated not only with epicardial coronary flow but also with perturbations in downstream microcirculatory flow at the level of coronary microvessels and the recognition of microvascular dysfunction could cause a paradigm shift in clinical practice [Shimokawa and Yasuda 2008]. Thrombus formation is a problem in many clinical situations, mainly cardiovascular diseases where platelets are also involved and in atherothrombotic disease since they support development of thrombus formation on atherosclerotic plaques eventually resulting in occlusion of vessels and cell death, exemplified by acute myocardial infarction [De Meyer et al. 2009].

Antithrombotic therapy is a cornerstone of cardiovascular medicine and randomised clinical studies have shown the benefit of aspirin both as a primary and a secondary prevention of ischemic events (myocardial infarction (MI), stroke and cardiovascular death) in patients with cardiovascular disease [Hansson. 2005]. Addition of a platelet ADP receptor inhibitor (clopidogrel) further reduced the incidence of ischemic events significantly and is the current standard for post-intervention care in patients undergoing percutanous coronary intervention (PCI) receiving coronary stents [A randomised, blinded, trial of clopidogrel versus aspirin in patients at risk of ischaemic events (CAPRIE), 1996]. However, most deaths resulting from acute myocardial infarction occur within 1 hour of its onset, and half of these occur before hospital admission. Thus, an effective pre-hospital treatment aiming at reducing ischemia and/or the downstream events of ischemia at the cellular level is highly warranted.

A subpopulation of patients undergoing PCI develop the no-reflow and slow-reflow phenomena, which are poorly understood complications of PCI in which diminished blood flow to distal vascular bed persists despite the treatment of the occlusive lesion from the epicardial coronary artery or arteries [Kiernan et al. 2009]. Pharmacologic adjuncts to PCI capable of impeding platelet aggregation or directly reducing microvascular spasm are the main therapeutic strategy for the treatment of no-reflow given the importance of both processes in the development of no-reflow pathophysiology. A range of pharmacologic adjuncts have been evaluated to limit no-reflow, including adenosine, diltiazem, nicardipine, verapamil, nitroprusside, glycoprotein antiplatelet medications and antiendothelins. However, large-scale, randomized, controlled trials are lacking to confirm their roles and define optimal regimens.

### The Endothelium and Cardiovascular Disease

The endothelium plays a crucial role in regulating vascular tone, growth, inflammatory response, coagulation, and platelet adhesion. Common conditions predisposing to atherosclerosis, such as dyslipidemia, hypertension, diabetes, and smoking are associated with endothelial dysfunction, which likely in part explains why these conditions are risk factors and promote the development, progression, and complications of atherosclerosis [Landmesser et al. 2005].

Accumulating clinical studies suggest an important pathophysiological role of endothelial dysfunction, as determined by impaired endothelium-dependent vasodilation, by demonstrating a close association of the degree of coronary or peripheral endothelial dysfunction with cardiovascular events [Landmesser et al. 2004]. Recently, endothelial dysfunction has been shown to predict future cardiovascular events in patients who have had an acute coronary syndrome [Fichtlscherer et al. 2004]. In addition, impaired flow-dependent, endothelium mediated vasodilation predicted the occurrence of in-stent restenosis in patients undergoing percutaneous coronary intervention in a recent prospective study [Patti et al. 2005].

Endothelial dysfunction may not only promote vascular inflammation, but conversely systemic inflammation can induce endothelial dysfunction [Hingorani et al. 2000]. This has been convincingly demonstrated in a recent large-scale study of 600 children with acute infection, who had a substantially impaired endothelium-dependent vasodilation during acute infections [Charakida et al. 2005].

Furthermore, inflammation induced endothelial dysfunction may provide, at least in part, an explanation for the recent observation that patients with rheumatoid arthritis, a systemic inflammatory disease, have a markedly increased risk for cardiovascular events [Solomon et al. 2003].

### Current Interventions to Improve Endothelial Function

### Life Style Changes

The recent INTERHEART study has underscored the important role of life style for cardiovascular risk [Yusuf et al. 2004]. Exercise training has been shown to improve endothelial function in patients with coronary disease or heart failure in the forearm, as well as in the coronary circulation [Hambrecht et al. 2000]. Adams et al. [Adams et al. 2005] have shown that exercise training reduces vascular oxidant stress and nicotinamide adenine dinucleotide phosphate oxidase activity in patients with coronary disease.

### Statins

Statins rapidly improve endothelium-dependent vasomotion in humans [Laufs et al. 2005]. In a recent study, simvastatin, but not ezetimibe, therapy improved endothelial function and increased EPCs in patients with CHF, suggesting that prolonged statin treatment can exert beneficial effects on endothelial function independent of lipid lowering [Landmesser et al. 2005].

### Inhibition of Angiotensin-Converting Enzyme Inhibitor and Angiotensin-1 Receptor Blockade

Angiotensin-converting enzyme (ACE) inhibition and angiotensin-1 receptor blockade have been shown to exert beneficial effects on endothelial function [Hornig et al. 2001], and recently angiotensin-1 receptor blockade has been shown to increase circulating EPCs [Bahlmann et al. 2005]. First studies suggest that combined statin and ACE inhibitor or angiotensin-1 receptor blocker treatment is more effective in improving endothelial function than single therapy [Joh et al. 2005]; however, more studies in different patient populations are needed to address the effect of combined treatment.

### BRIEF DESCRIPTION OF DRAWINGS

FIG. 1: Recording haemostatic activity using TEG assay.

FIG. 2: MultiPlate continuously records platelet aggregation. The increase of impedance by the attachment of platelets onto the Multiplate sensors is transformed to arbitrary aggregation units (AU) and plotted against time.

### SUMMARY OF THE INVENTION

Prostacyclin PGI2 has been reported to be one of the most potent endogenous platelet inhibitors existing [Szczeklik et al. 1978], in addition to its well described endothelial modulating effects, which may explain why PGI2 to date not have been administered to patients undergoing PCI.

Surprisingly, the present inventors have found that prostacyclin does not affect platelet aggregation in whole blood as evaluated by MULTIPLATE and, furthermore, prostacyclin does not impair thrombus formation as evaluated by viscoelastical haemostatic assays, such as thrombelastography (TEG). Hereby, the effect on the endothelium of PGI2 can be employed in conjunction with treatment with antithrombotic agents such as Aspirin (ASA), platelet ADP—and a platelet inhibitor, such as GPIIb/IIIa receptor antagonists without increased risk of bleeding in this high-risk population.

The inventors therefore propose that administration of endothelial modulators such as prostacyclin will target a key component of the acute coronary syndrome (ACS), i.e. the dysregulated endothelium, not currently addressed when treating of these patients with PCI alone. It is envisaged that the administration of prostacyclin should be performed in combination with existing treatments with antithrombotic agents, preferably with one or more of administration of a a platelet inhibitor, such as GPIIb/IIIa receptor antagonists or endovascular methods and/or surgical procedures such as percutaneous coronary intervention.

Furthermore, a combination of compounds that target both the endothelium and the platelets to obtain a synergistic effect of the compounds as compared to only targeting either the platelets or the endothelium is an aspect of the present invention. Also, it is an aspect that by combining the treatments, a lower level/dosage of the compound(s) to be administered may be required with the advantage of reduced risk of possible adverse events.

The theory behind the current invention is that endothelial dysregulation is a key factor in the pathogenesis of acute coronary syndrome (ACS) contributing to the adhesion of platelets and leukocytes resulting in further activation of the endotelium as well as development of thrombuses that may occlude the vessels. Furthermore, it is envisaged that an integral part of the endothelial dysregulation is a locally reduced production and/or release of prostacyclin from the endothelium in the microvascular bed surrounding the occlusion.

It is envisaged that the administration of prostacyclin should be performed in combination with existing treatments with antithrombotic agents, preferably with one or more of administration of a a platelet inhibitor, such as GPIIb/IIIa receptor antagonist or endovascular and/or surgical methods such as percutaneous coronary intervention.

The present invention relates in a first aspect to pharmaceutical compositions comprising one or more of any of the compounds mentioned herein below, such as one compound, such as at least two compounds, such as at least three compounds. When using more than one compound, the compounds may be selected from the same group of compounds, or more preferably the at least two compounds may be selected from different groups of compounds. Accordingly, in one embodiment one compound is a compound capable of modulating/preserving the endothelial integrity or a compound capable of inhibiting platelet aggregation. Thus another aspect of the invention relates to a pharmaceutical composition comprising one or more compounds capable of modulating/preserving the endothelial integrity for use in the treatment and/or prevention of ischemic events in human patients being at increased risk of development of an ischemic event such as an acute myocardial infarction and/or no-reflow phenomena and/or ischemia-reperfusion injury.

In another embodiment, one compound is a compound capable of modulating/preserving the endothelial integrity and the other compound is a compound capable inhibiting platelet aggregation.

Preferably the one or more compounds are a platelet inhibitor and a compound capable of modulating/preserving the endothelial integrity, more preferably an antithrombotic compound even more preferably PGI2 and a GPIIb/IIIa inhibitor.

Another aspect of the invention relates to the use of a pharmaceutical composition as described herein for treatment and/or prevention of ischemic events in human patients being at increased risk of development of an ischemic event such as an acute myocardial infarction and/or no-reflow phenomena and/or ischemia-reperfusion injury, preferably said patients are ACS patients. Patients being at increased risk of developing an ischemic event such as an acute myocardial infarction and/or no-reflow phenomena and/or ischemia-reperfusion injury will in at least some cases already be undergoing treatment for cardiovascular disease such as acute myocardial infarction. Any such treatments are comprised within the scope of the present invention.

Still another aspect of the invention relates to the use of the pharmaceutical composition as described herein for prevention or treatment of ischemic events in human patients being at increased risk of development of an ischemic event such as an acute myocardial infarction and/or no-reflow phenomena and/or ischemia-reperfusion injury, preferably said patients are ACS patients.

Another aspect of the invention relates to a composition comprising one or more, such as two or more compounds selected from the group consisting of compounds capable of modulating/preserving the endothelial integrity and/or platelet inhibitors, for use in treatment and prevention of ischemic events in human patients being at increased risk of development of an ischemic event such as an acute myocardial infarction and/or no-reflow phenomena and/or ischemia-reperfusion injury, preferably said patients are ACS patients.

Another aspect of the invention relates to the use of one or more compounds, such as two or more compounds selected from the group consisting of compounds capable of modulating/preserving the endothelial integrity in the manufacture of a medicament for the treatment or prevention of ischemic events in human patients being at increased risk of development of an ischemic event such as an acute myocardial infarction and/or no-reflow phenomena and/or ischemia-reperfusion injury.

Yet another aspect of the invention relates to a method of treatment of cardiovascular ischemia comprising administering to a human subject in need thereof one or more, such as two or more compounds selected from the group consisting of platelet inhibitors and/or compounds capable of modulating/preserving the endothelial integrity.

Still another aspect of the invention relates to a method of treating or preventing ischemic events in human patients being at increased risk of development of an ischemic event such as an acute myocardial infarction and/or no-reflow phenomena and/or ischemia-reperfusion injury comprising administering one or more, such as two or more compounds capable of modulating/preserving the endothelial integrity.

Additional aspects of the present invention and particular embodiments will be apparent from the description below, as well from the appended claims.

### DETAILED DESCRIPTION OF THE INVENTION

Interventions aiming at modulating/preserving endothelial integrity (keeping the endothelium in a quiescent inactivated anti-coagulant state, eg. by the administration of endothelial modulators) will reduce endothelial activation and improve endothelial integrity and therefore prohibit and/or reduce development of thrombus formation in the microvasculature which will prevent and/or limit cardiovascular ischemia and/or no-reflow phenomena and/or reperfusion-ischemia injury.

Also, interventions aiming at modulating/preserving endothelial integrity in combination with platelet inhibitors will reduce endothelial activation and improve endothelial integrity, reduce platelet aggregation and therefore prohibit and/or reduce development of thrombus formation in the microvasculature which will prevent and/or limit cardiovascular ischemia and/or no-reflow phenomena and/or reperfusion-ischemia injury.

Accordingly, the present invention relates to compounds for a new treatment modality for patients having cardiovascular disease having or being at increased risk of development of an ischemic event such as an acute myocardial infarction and/or no-reflow phenomena and/or ischemia-reperfusion injury.

Given the above-mentioned association between an ischemic event such as an acute myocardial infarction and/or no-reflow phenomena and/or ischemia-reperfusion injury. and/or endothelial activation and/or endothelial dysregulation, interventions that modulate and/or preserve endothelial integrity by keeping the endothelium in a quiescent inactivated anti-coagulant state (endothelial modulators, described herein below); in combination with standard treatment of acute cardiovascular ischemic events such as:
1. Platelet inhibitors (Aspirin (ASA), Thienopyridins, GPIIb/IIIa inhibitors)
2. Parenteral anticoagulants (UFH, bivalirudin, enoxaparin, and fondaparinux)
3. PCI, PCI with thrombus aspiration, PCI with stents
4. Verapamil
5. Adenosine
6. Sodium nitroprusside
7. Nitroglycerin
8. Pressor drugs/rescue drugs including but not limited to Epinephrine, norepinephrine, dopamine and dobutamine
9. Beta-blockers
   should be used to prevent and/or cure imminent and/or manifest cardiovascular disease in patients having or being at increased risk of development of an ischemic event such as an acute myocardial infarction and/or no-reflow phenomena and/or ischemia-reperfusion injury. These human patients may have any condition associated with cardiovascular disease (conditions suitable for the invention, described herein below).

### DEFINITIONS

The term “modulating/preserving endothelial integrity” is intended to mean pharmacological treatment aiming at maintaining the endothelium in a quiescent inactivated anti-coagulant state. Thus a “compound capable of modulating/preserving endothelial integrity” is intended to mean any compound that may assist in maintaining/inducing the endothelium in a quiescent inactivated anti-coagulant state.

The term “atherosclerosis” is intended to mean the condition in which an artery wall thickens as the result of a build-up of fatty materials such as cholesterol. It is a syndrome affecting arterial blood vessels, a chronic inflammatory response in the walls of arteries, in large part due to the accumulation of macrophage white blood cells and promoted by low-density lipoproteins (plasma proteins that carry cholesterol and triglycerides) without adequate removal of fats and cholesterol from the macrophages by functional high density lipoproteins (HDL).

The term “acute coronary syndrome (ACS)” is intended to mean a set of signs and symptoms related to the heart. ACS is compatible with a diagnosis of acute myocardial ischemia-but it is not characteristic of the diagnosis. The sub-types of acute coronary syndrome include unstable angina (UA, not associated with heart muscle damage), and two forms of myocardial infarction (MI, heart attack), in which heart muscle is damaged. These types are named according to the appearance of the electrocardiogram (ECG/EKG) as non-ST segment elevation myocardial infarction (NSTEMI) and ST segment elevation myocardial infarction (STEMI).

The term “ischemic event” is intended to mean: when local tissue demand for energy substrates is not met by supply, with hypoxia (low oxygen) being an important component of an ischemic insult.

The term “ischemia” is intended to mean a restriction in blood supply, generally due to factors in the blood vessels, with resultant damage or dysfunction of tissue.

The term “hypoxia” is intended to mean a pathological condition in which the body as a whole (generalized hypoxia) or a region of the body (tissue hypoxia) is deprived of adequate oxygen supply.

The term “no-reflow phenomena” is intended to mean the failure of blood to reperfuse an ischemic area after the physical obstruction has been removed or bypassed.

The term “slow-reflow phenomena” is intended to mean a complication where diminished blood flow to distal vascular bed persists despite the treatment of the occlusive lesion from the epicardial coronary artery or arteries.

The term “ischemia-reperfusion injury” is intended to mean damage to tissue caused when blood supply returns to the tissue after a period of ischemia. The absence of oxygen and nutrients from blood creates a condition in which the restoration of circulation results in inflammation and oxidative damage through the induction of oxidative stress rather than restoration of normal function.

The term “endothelial dysfunction” is intended to mean a local or systemic pathological state of the endothelium (the inner lining of our blood vessels) and can be broadly defined as an imbalance between vasodilating and vasoconstricting substances as well as pro- and anticoagulant substances produced by (or acting on) the endothelium.

The term “acute myocardial infarction” is intended to mean: Myocardial infarction (MI) or acute myocardial infarction (AMI), commonly known as a heart attack, is the interruption of blood supply to part of the heart, causing some heart cells to die.

The term “percutanuous coronary intervention (PCI)” also known as coronary angioplasty or simply angioplasty or percutaneous transluminal coronary angioplasty (PTCA), is intended to mean a therapeutic procedure used to treat the stenotic (narrowed) coronary arteries of the heart found in coronary heart disease.

The term “cardiovascular ischemia/cardiovascular ischemic event” is intended to mean: when local tissue demand for energy substrates is not met by supply, with hypoxia (low oxygen) being an important component of an ischemic insult. Hypoxia is a reduction in oxygen delivery below tissue demand, whereas ischemia is a lack of perfusion, characterized not only by hypoxia but also by insufficient nutrient supply.

The term “antiaggregatory” is intended to mean a lower than normal ability of the platelets to interact in the clot building process secondary to administration of compounds and/or variants that inhibit the platelets ability to aggregate.

The term “antithrombotic” is also intended to mean a lower than normal ability of the platelets to interact in the clot building process secondary to administration of compounds and/or variants that inhibit and/or decreases the platelets ability to aggregate and inhibit the platelets ability to form clots (thrombus formation).

The terms “antiaggregatory” and “antithrombotic” is used interchangeably and refers to the effect of compound(s) that reduces the platelets ability to interact in the clot building process and hence form thrombi.

The term “homeostasis” refers to the body's ability to regulate physiologically its inner environment to ensure its stability. An inability to maintain homeostasis may lead to death or a disease.

The term “TIMI” stands for ‘Thrombolysis In Myocardial Infarction’ and is the name of a study group coordinating several trials, particularly focusing on percutaneous coronary intervention, thrombolysis as well as cardiovascular disease in general. The TIMI classification/bleeding scale defines bleeding as:
Major Intracranial bleeding; overt bleeding with a decrease in hemoglobin ≧5 g/dl or a decrease in hematocrit ≧15%
Minor Spontaneous gross hematuria; spontaneous hematemesis; observed bleeding with decrease in hemoglobin ≧3 g/dl but ≦15%
Insignificant Blood loss insufficient to meet criteria listed above

The term “GUSTO” stands for Global Use of Strategies to Open Occluded Coronary Arteries. The GUSTO classification/bleeding scale defines bleeding as:
Severe Deadly bleeding; intracerebral bleeding or substantial hemodynamic compromise requiring treatment
Moderate Bleeding requiring transfusion
Mild Other bleeding not requiring transfusion or causing hemodynamic compromise

The term “Mulitplate” refers to an applied platelet aggregometer/platelet function analyzer utilizing the analysis of whole blood. Whole blood is the physiological environment where platelet function takes place in vivo, and the use of whole blood for in-vitro testing eliminates the need for time-consuming centrifugation steps required for Born aggregation measurements. The use of a small amount of whole blood (0.3 ml per test) allows the differentiated assessment of platelet function without drawing large amounts of blood (as required for analysis of Born aggregation) and facilitates the analysis of blood from children and also in experimental settings.

A “subject” includes humans and other mammals, and thus the methods are applicable to both human therapy and veterinary applications, in particular to human therapy. The term “mammal” includes humans, non-human primates (e.g. baboons, orangutans, monkeys), mice, pigs, cows, goats, cats, dogs, rabbits, rats, guinea pigs, hamsters, horse, monkeys, sheep or other non-human mammal.

“Treatment”, as used in this application, is intended to include both prevention of an expected development or treatment of an established ischemic event in patients being at increased risk of developing an ischemic event such as an acute myocardial infarction and/or no-reflow phenomena and/or ischemia-reperfusion injury.

“Reperfusion injury” as used herein refers to damage to tissue caused when blood supply returns to the tissue after a period of ischemia. The absence of oxygen and nutrients from blood creates a condition in which the restoration of circulation results in inflammation and oxidative damage through the induction of oxidative stress rather than restoration of normal function.

“Patients” as used herein refers to patients being at increased risk of development of an ischemic event such as an acute myocardial infarction and/or no-reflow phenomena and/or ischemia-reperfusion injury. These patients may have any cardiovascular disease particularly preferred are patients with ACS. “Patients” are also intended to include any cardiovascular patients about to undergo, undergoing or having undergone endovascular and/or surgical procedures such as PCI, coronary angiography, Coronary artery bypass surgery (CABG) or thrombolytic therapy.

### Embodiments

As described herein above, a main aspect of the invention relates to compounds for treatment that protects the endothelium, prevent pathologic thrombus formation in the microcirculation thus may be related to minimizing or preventing, ischemic events and/or no-reflow phenomena and/or ischemia-reperfusion injury and, hence, death in cardiovascular/critically ill patients by administration of agents modulating/preserving endothelial integrity in some embodiments in combination with platelet inhibitors and pharmaceutical compositions comprising one or more, such as two or more of any of the compounds mentioned.

In one embodiment said compounds are preferably compounds that modulate/preserve endothelial integrity and are more preferably selected from one or more of the groups described herein below.

In the following, names of compounds of relevance for the present invention are listed. Trade names covering any of the herein mentioned compounds are also of relevance for the present invention.

### Agents Modulating/Preserving Endothelial Integrity

The endothelium maintains under physiological conditions a normal vascular function by regulating the balance between vasodilator and vasoconstrictor mediators and by regulating the expression/release of adhesion receptors and pro- and anticoagulant molecules at/from the endothelium. Endothelial modulators encompass any agent that affects the endothelium to either maintain or develop into a non-activated quiescent state, which optimally preserves and ensures vascular integrity. In a state with vascular integrity, the endothelium exerts anti-inflammatory and anti-thrombotic properties down-regulating and counteracting platelet activation through the generation of PGI2 (prostaglandin 12, prostacyclin) and through the production of ADPase, the latter catalyzing the degradation of ADP. Endothelial cells can also prevent the activation of the coagulation cascade by expressing surface molecules with anticoagulant properties such as heparan sulfate, dermatan sulfate, tissue factor pathway inhibitor (TFPI), protein S (PS) and thrombomodulin (TM). Endothelial cells express plasminogen, tissue-type plasminogen activator (tPA), urokinase-type plasminogen activator (uPA), urokinase-type plasminogen activator receptor (uPAR) as well as membrane-associated plasminogen activator binding sites, thus favouring the generation of plasmin, and they express endothelial protein C receptor (EPCR), which enhances the anticoagulant activity.

The endothelial modulators may be selected from any of the classes of compounds (1-12) described below:
1. Compounds such as PGI2 (flolan), PGX, prostacyclin (Epoprostenol) or variants thereof, such as beraprost sodium, epoprostenol sodium, iloprost, iloprost in combination with bosentan, iloprost in combination with sildenafil citrate, treprostinil, pegylated treprostinil, treprostinil diethanolamine and treprostinil sodium. Further compounds are 2-{4-[(5,6-diphenylpyrazin-2-yl)(isopropyl)amino]butoxy}-N-(methylsulfonyl)acetamide, {4-[(5,6-diphenylpyrazin-2-yl)(isopropyl)amino]butoxy}acetic acid, 8-[1,4,5-triphenyl-1H-imidazol-2-yl-oxy]octanoic acid, isocarbacyclin, cicaprost, [4-[2-(1,1-Diphenylethylsulfanyl)-ethyl]-3,4-dihydro-2H-benzo[1,4]oxazin-8-yloxy]-acetic acid N-Methyl-d-glucamine, 7,8-dihydro-5-(2-(1-phenyl-1-pyrid-3-yl-methiminoxy)-ethyl)-a-naphthyloxyacetic acid, (5-(2-diphenylmethyl aminocarboxy)-ethyl)-a-naphthyloxyaceticacid, 2-[3-[2-(4,5-diphenyl-2-oxazolyl)ethyl]phenoxy]acetic acid, [3-[4-(4,5-diphenyl-2-oxazolyl)-5-oxazolyl]phenoxy]acetic acid, bosentan, 17[alpha], 20-dimethyl-[DELTA]6,6a-6a-carba PGI1, and 15-deoxy-16[alpha]-hydroxy-16[beta], 20-dimethyl-[DELTA]6,6a-6a-carba PGI1, pentoxifylline (1-{5-oxohexyl}-3,7-dimethylxanthine).
   Trade names for prostacyclins include, but are not limited to: flolan, remodulin, and ventavis.
2. A combination of prostacyclin or a prostacyclin analogue and endothelin receptor antagonist may improve the safety profile of prostacyclin therapy by reducing potential side effects of prostacyclin such as jaw pain, headache and hypotension.
3. Compounds with modulating/preserving endothelial effects such as nitric oxide (also Endothelium Derived Relaxing Factor) produced by healthy endothelial cells induce vasodilatation and favours an anti-adhesive and anti-inflammatory phenotype of the endothelium through a rise in cytosolic cGMP [Cines et al 1998; Zardi et al 2005].
4. CD39 and CD73 are vascular membrane-bound ecto-nucleotidases expressed at the luminal surface of healthy endothelial cells. They hydrolyze extracellular plasma ATP and ADP and thereby inhibit nucleotide mediated platelet activation [Atkinson et al 2006; Colgan et al 2006]. In addition to platelet inhibition, soluble CD39 and CD73 agonists inhibit endothelial cell apoptosis and activation [Goepfert et al 2000] and prevent hypoxia induced vascular leakage [Thompson et al 2004].
5. Compounds involved in redox control of endothelial functions such as: L-Arginine and tetrahydrobiopterin, Antioxidants (Ascorbate, Glutathione, α-tocopherol, ubiquinol-10, Probucol), Iron chelators, and Polyphenols.
6. Clinical drugs involved in redox control of endothelial functions such as: HMG-CoA reductase inhibitors (Fluvastatin, Lovastatin, Pravastatin, Simvastatin), Angiotensin-receptor antagonists and ACE inhibitors (Captopril, Zofenopril, Enalapril, Ramipril, Quinapril, Perindopril, Lisinopril, Benazepril, Fosinopril, Casokinins, lactokinins), Peroxisome proliferator-activated receptors (PPARs), NADPH oxidase, Xanthine oxidase, PETN, Heparan sulfates (PI-88), heparan sulfate mimetics, Activators of oxidized/heme-free sGC (BAY 58-2667), and Anti-PECAM/SOD.
7. Honokiol, a biphenyl neolignan isolated from Hou pu, the cortex of Magnolia officinalis.
8. Compounds that directly modulate endothelial barrier function through modulating effects on sphingosine-1-phosphate (S1P)-receptors (eg.: FTY720, AA-R, AAL-S, KRP-203, AUY954, CYM-5442, SEW2871, W146, W140, VPC44116, VPC23019, JTE-013) [Marsolais et al 2009].
9. Antibodies and/or other molecules against/antagonizing histones that through their inhibition diminishes histone-mediated endothelial damage and/or microthrombi formation and/or fibrin deposition [Xu et al 2009].
10. Compounds enhancing the natural anticoagulant pathways and hence protecting the endothelium such as but not exclusively: Protein C pathway (Activated protein C (APC, Drotrecogin alfa), protein C, compounds that either mimics and/or protects from degradation and/or enhances soluble thrombomodulin and/or EPCR and/or protein S), Antithrombin III (ATIII) (or ATIII like compounds and/or compounds that enhance ATIII function) and tissue factor pathway inhibitor (TFPI) (or TFPI compounds and/or compounds that enhance TFPI function).
11. Compounds that maintain and/or promote Gβy function and/or signalling following endothelial PAR activation to ensure reannealing of adherens junctions opened following inflammatory PAR mediated activation of Gα [Knezevic et al 2009]
12. Defibrotide is a polydisperse oligonucleotide with antiatherosclerotic anti-inflammatory, anti-ischaemic, pro-fibrinolytic and antithrombotic actions, currently used in the treatment of various cardiovascular disorders, and especially in endothelial complications of allogeneic stem-cell transplantation. It upregulates the release of prostacyclin (PGI2) and prostaglandin E2 (PGE2), reduces concentrations of leukotriene B4, inhibits monocyte superoxide anion generation, stimulates expression of thrombomodulin in human vascular endothelial cells (ECs), and modulates platelet activity, the latter due, at least partly, to the increased release of prostaglandins (PGI2 and PGE2). Another important antithrombotic mechanism induced by defibrotide is the activation of the fibrinolytic system, which is mainly attributed to the increase of tissue plasminogen activation (t-PA) [Morabito 2009].

Various other potential target sites to modulate the endothelial function, activation state and integrity are given in Table 1, below.

TABLE 1
Potential endothelial modulating target sites

| Targets | | Compound |
|---|---|---|
| 1. | Inhibition of Rho-kinase | Fasudil |
| | | Y-27632 |
| 2. | Inhibition of PARP | PJ-34 |
| | | INO 1001 |
| | | 3-Aminobenzamide |
| 3. | Inhibition of PTPase | Bis(malotalo) oxovanadium |
| 4. | Activation of Akt | Demethylasterriquinone |
| 5. | Activation of PKA | 8-Br-cAMP |
| 6. | Inhibition of caveolin | Daidzein |
| 7. | estrogen-receptor (ER) agonist | 17-beta-Estradiol |
| 8. | Activation of | |
| | PPAR alpha | Fibrates |
| | PPAR gamma | Thiazolidinediones |
| | PPAR delta | GW 07242 |
| 9. | Inhibition of CETP | Torcetrapib |
| | | CETi-1 vaccine |
| 10. | Activation of lipoprotein lipase | NO-1886 |
| 11. | Activation of S1P | FTY720 |
| 12. | Activation of transketolase | Benfotiamine |
| 13. | Inhibition of GGT | GGTI-298 |
| 14. | Inhibition of epoxide hydrolase | 1-Cyclohexyl-3-dodecylurea |
| | | N,N′-Dicyclohexylurea |
| | | N,N′-Adamantanyl-N′-dodecanoic |
| | | urea |
| 15. | Activation of ACE 2 | AVE 0991 |
| 16. | Inhibition of JAK | AG-490 |
| | | WHI-P154 |

Prostacyclin, a metabolite of arachidonic acid, is a naturally occurring prostaglandin with potent vasodilatory activity and inhibitory activity of platelet aggregation, released by healthy endothelial cells. Prostacyclin performs its function through a paracrine signalling cascade that involves G protein-coupled receptors on nearby platelets and endothelial cells. In the clinical setting, Epoprostenol (prostacyclin analogue) has 2 major pharmacological actions: (1) direct vasodilation of pulmonary and systemic arterial vascular beds, and (2) inhibition of platelet aggregation. Epoprostenol is indicated for the long-term intravenous treatment of primary pulmonary hypertension and pulmonary hypertension associated with the scleroderma spectrum of disease in NYHA Class III and Class IV patients who do not respond adequately to conventional therapy. The antiaggregatory effect of prostacyclin analogs on platelets is mediated by the Gas protein-coupled receptor (prostacyclin receptor, IP) that is activated upon prostacyclin analog binding. This activation signals adenylyl cyclase to produce cAMP, which in turn activates Protein Kinase A to decrease free intracellular calcium concentrations. The rise in cAMP directly inhibits platelet activation (secretion and aggregation) and counteracts increases in cytosolic calcium resulting from platelet activation by agonists such as thrombin, ADP, TXA2, PAF, collagen and 5-HT [Bihari et al, 1988; Schereen et al, 1997; Xing et al 2008].

The modulating/preserving effect on endothelial integrity is mediated by binding of prostacyclin analog to endothelial prostacyclin receptors with ultimate rise in cytosolic cAMP and Protein Kinase A activation. This leads to smooth muscle relaxation and vasodilatation with improved microvascular perfusion and “cytoprotection” through stabilization of lysozomal and cell membranes with reduced inflammation. It also favours an anti-coagulant, anti-adhesive, anti-apoptotic and anti-inflammatory phenotype of the endothelium, less likely to support coagulation, leukocyte adhesion/migration and inflammation [Zardi et al 2005; Zardi et al 2007].

In a preferred embodiment the compound capable of modulating/preserving the endothelial integrity has a half time of less than 4 hours (such as Treprostinil), preferably less than 1 hours (such as Beraprost (35-40 min)), more preferably less than ½ hour (such as Iloprost (20-30 min)), even more preferably less than 5 min (such as PGI2 (flolan) or prostacyclin (Epoprostenol) (0, 5-3 min))

### Platelet Inhibitors

As described herein above, a main aspect of the invention relates to compounds for treatment that protects the endothelium, prevent pathologic thrombus formation in the microcirculation thus may be related to minimizing or preventing, ischemic events and/or no-reflow phenomena and/or ischemia-reperfusion injury and, hence, death in cardiovascular/critically ill patients by administration of agents modulating/preserving endothelial integrity in combination with platelet inhibitors and pharmaceutical compositions comprising one or more of any of the compounds mentioned.

Thus, in one embodiment said platelet inhibitors are preferably selected from one or more of the groups described herein below.

Platelet inhibitors are compounds that interfere with platelet activation (including adhesion, secretion), aggregation and ultimate platelet-fibrin clot formation. Consequently, platelet activation including secretion of alpha, dense, lysosomal and other granules are reduced or inhibited. Also, exposure of negatively charged phosphatidylserine on the platelet surface is reduced or inhibited. Furthermore, activation of the GPIIb/IIIa receptor, being the final common pathway for activation by the thromboxane receptor, ADP receptor and PAR receptors is prevented or limited. In addition, several platelet receptors and/or molecules are reduced or inhibited.

Any agent that reversibly or irreversibly reduces and more preferably inhibits platelet activation/aggregation by blocking sites on the platelet surface or capable of intracellular inhibition can be used as the platelet inhibitor in the present invention.

Platelet inhibitors according to present invention may include any agent that is intended to be used as an antithrombotic or antiaggregatory agent. Any agent that reversibly or irreversibly reduces, modulates and/or more preferably inhibits platelet activation/aggregation by blocking sites on the platelet surface or capable of intracellular inhibition of pathways that mediates platelet activation can be used as the platelet inhibitor in the present invention.

A non-exhaustive list of examples of platelet inhibitors for the prevention or treatment of cardiovascular disease in patients having or being at increased risk of development of an ischemic event such as an acute myocardial infarction and/or no-reflow phenomena and/or ischemia-reperfusion injury encompass the following:
1. Compounds inhibiting the platelet GPIIb/IIIa receptor such as: abciximab, eptifibatide (integrilin), tirofiban, orbofiban, xemilofiban, lamifiban, XJ757, DUP728, XR299, linear or novel cyclic RGD peptide anlogs, cyclic petides, peptidomimetics inhibiting this receptor and the like, and mixtures hereof and other compounds.
   In a particular embodiment the compound inhibiting the platelet GPIIb/IIIa receptor is administered together with a prostacyclin or a prostacyclin analog, see below.
2. Compounds inhibiting the platelet ADP receptor (P2Y12) such as: a Thienopyridin for example Ticlopidine (Ticlid), Clopidogrel (Plavix), Prasugrel, AR-C69931 MX, AZD6140, cangrelor, ticagrelor and other compounds inhibiting this receptor.
   In a particular embodiment the compound inhibiting the platelet ADP receptor (P2Y12) is administered together with a prostacyclin or a prostacyclin analog, see below.
3. Compounds inhibiting the platelet P2Y₁ receptor such as: MRS2500, MRS2298, MRS2496, A2P5P, A3P5P, ATP, 2-MeSATP, and 2-CIATP.
   In a particular embodiment the compound inhibiting the platelet receptor (P2Y1) is administered together with a prostacyclin or a prostacyclin analog, see above.
4. Compounds inhibiting the platelet COX1 and/or COX2 pathways such as
   a. COX inhibitors which have the ability to inhibit as well COX1 as COX2, such as
      i. Salicylates selected from the group consisting of Acetylsalicylic acid (Aspirin), Amoxiprin, Benorylate/Benorilate, Choline magnesium salicylate, Diflunisal, Ethenzamide, Faislamine, Methyl salicylate, Magnesium salicylate, Salicyl salicylate and Salicylamide;
      ii. Arylalkanoic acids selected from the group consisting of Diclofenac, Aceclofenac, Acemethacin, Alclofenac, Bromfenac, Etodolac, Indomethacin, Nabumetone, Oxametacin, Proglumetacin, Sulindac and Tolmetin;
      iii. 2-Arylpropionic acids (profens) selected from the group consisting of Ibuprofen, Alminoprofen, Benoxaprofen, Carprofen, Dexibuprofen, Dexketoprofen, Fenbufen, Fenoprofen, Flunoxaprofen, Flurbiprofen, Ibuproxam, Indoprofen, Ketoprofen, Ketorolac, Loxoprofen, Naproxen, Oxaprozin, Pirprofen, Suprofen and Tiaprofenic acid;
      iv. N-Arylanthranilic acids (fenamic acids) selected from the group consisting of Mefenamic acid, Flufenamic acid, Meclofenamic acid and Tolfenamic acid;
      v. Pyrazolidine derivatives selected from the group consisting of Phenylbutazone, Ampyrone, Azapropazone, Clofezone, Kebuzone, Metamizole, Mofebutazone, Oxyphenbutazone, Phenazone and Sulfinpyrazone;
      vi. Oxicams selected from the group consisting of Piroxicam, Droxicam, Lornoxicam, Meloxicam and Tenoxicam;
   b. COX inhibitors which are specific for inhibition of COX2 such as Celecoxib, Etoricoxib, Lumiracoxib, Parecoxib, Rofecoxib, Valdecoxib, Nimesulide, Licofelone and Omega-3 fatty acids.
   In a particular embodiment the compound inhibiting COX is administered together with a prostacyclin or a prostacyclin analog, see above.
5. Compounds inhibiting thromboxane-synthase (TX-synthase) such as flavonoids and thromboxane receptor (TP)-antagonists, such as SQ29548, Bay u 3405, or BM 13.177.
   In a particular embodiment the compound inhibiting thromboxane-synthase (TX-synthase) and/or thromboxane receptor (TP)-antagonists is administered together with a prostacyclin or a prostacyclin analog, see above.
6. Compounds inhibiting adenosine uptake in the platelets such as dipyramidol, Persantin, Asasantin, Aggrenox and other compounds with a similar mode of action.
   In a particular embodiment the compound inhibiting adenosine uptake in the platelets is administered together with a prostacyclin or a prostacyclin analog, see above.
7. Compounds inhibiting the platelet GPIb receptor, such as mAB lb-23, mAB 6B4, R9alpha557 peptide, aurintricarboxylic acid (ATA), crotalin, agkistin, peptide (Trp-Ile-Arg-Arg-Pro-Phe-Phe-Pro-Phe) from alpha B-crystallin.
   In a particular embodiment the compound inhibiting the platelet GPIb receptor is administered together with a prostacyclin or a prostacyclin analog, see above.
8. Compounds inhibiting the platelet GPVI receptor, such as EXP3179, triplatin-1 and -2, JAQ1, mAB 10B12, mAB 1C3, mAb 12G1.
   In a particular embodiment the compound inhibiting the platelet GPVI receptor is administered together with a prostacyclin or a prostacyclin analog, see above.
9. Compounds inhibiting the PAR receptors such as thrombin inhibitors, heterocycle-based peptide-miimetic antagonists of PAR-1, RWJ-56110 and RWJ-58259, SCH 79797, SCH 203099, and PAR4 antagonists such as trans-cinnamoyl-YPGKF-amide (tc-Y—NH(2)) and palmitoyl-SGRRYGHALR-amide (P4pal10), PAR-2 antagonist ENMD-1068, PAR2 monoclonal antibody SAM-11.
   In a particular embodiment the compound inhibiting the PAR receptors is administered together with a prostacyclin or a prostacyclin analog, see above.
10. Phosphodiesterase inhibitor PDE3 such as Cilostazol with therapeutic focus on increasing cAMP. An increase in cAMP results in an increase in protein kinase A (PKA), which is directly related with an inhibition in platelet aggregation.
   In a particular embodiment the Phosphodiesterase inhibitor is administered together with a prostacyclin or a prostacyclin analog, see above.
11. Nitroaspirin (NCX4016) an aspirin that can release NO.
   In a particular embodiment nitroaspirin is administered together with a prostacyclin or a prostacyclin analog, see above.
12. A compound of albumin conjugated with polyethylene glycol (PEG).
   In a particular embodiment the albumin conjugated with PEG inhibitor is administered together with a prostacyclin or a prostacyclin analog, see above.
13. A compound of haemoglobin conjugated with polyethylene glycol, a compound that besides its platelet inhibitory function also improves oxygenation of the microvasculature, such as but not exclusively MP40X (Hemospan, polyethylene glycol-hemoglobin complexes)
   In a particular embodiment the hemoglobin conjugated to PEG is administered together with a prostacyclin or a prostacyclin analog, see above.
14. Antibodies and/or inhibitors of C-type lectin-like receptor 2 (CLEC-2) [May et al 2009]
   In a particular embodiment the antibodies/inhibitors of CLEC-2 is administered together with a prostacyclin or a prostacyclin analog, see above.
15. High-energy glycolitic metabolites like fructose-1,6-bisphosphate (FBP) [de Oliveira et al]
   In a particular embodiment the FBP is administered together with a prostacyclin or a prostacyclin analog, see above.

In a preferred embodiment the platelet inhibitor has a half time of less than 3 hours (such as eptifibatide), preferably less than 2.5 hours (such as tirofiban), more preferably less than 1 hour (such as abciximab). In a preferred embodiment a compound inhibiting the platelet GPIIb/IIIa receptor is administered. Tirofiban is an example of a preferred compound. Eptifibatide is an example of a most preferred compound.

In another preferred embodiment the platelet inhibitor has a half time of less than 12 hours (such as Ticlopidine and prasugel), preferably less than 8 hours (such as Clopidogrel), more preferably about 3-5 min (such as cangrelor). Another preference is in the reversibility of the ADP receptor inhibition: Ticagrelor is an example of a compound that blocks the receptor in a reversible manner and Ticagrelor is for this reason preferable. Thus in an equally preferred embodiment a compound inhibiting the platelet ADP receptor (P2Y12) is administered.

In another preferred embodiment the platelet inhibitor is a COX inhibitor, such as an NSAID even more preferably aspirin.

Other preferred platelet inhibitors are Phosphodiesteraseinhibitors such as Cilostazol, preferably Dipyrimidole, even more preferably Triflusal.

Other preferred platelet inhibitors are Ditazole and Cloricromen.

In regards to the half lives/half times of the herein mentioned compounds: the half time depends on the administration form and/or the dosage. In general, intravenous administration is preferred.

### Combinations

As described herein above compounds that modulate and/or preserve endothelial integrity by keeping the endothelium in a quiescent inactivated anti-coagulant state (endothelial modulators) may be administered in combination with any one or more form of standard treatment of acute cardiovascular ischemic events including but not limited to
1. Platelet inhibitors (described herein above in detail including Aspirin (ASA), Thienopyridins, GPIIb/IIIa inhibitors);
2. Parenteral anticoagulants (unfractioned heparin (UFH), bivalirudin, enoxaparin, and fondaparinux);
3. Verapamil;
4. Adenosine;
5. Sodium nitroprusside;
6. Nitroglycerin;
7. Pressor drugs/rescue drugs including but not limited to Epinephrine, norepinephrine, dopamine and dobutamine
8. Beta-blockers;
9. Percutaneous coronary intervention (PCI), PCI with thrombus aspiration, PCI with stents.

Thus the invention relates to any combination of one or more, such as two or more compounds capable of modulating/preserving the endothelial integrity with any of the (classes of) compounds mentioned above (platelet inhibitors, parenteral anticoagulants, Verapamil; adenosine, Sodium nitroprusside, Nitroglycerin, Beta-blockers and Pressor drugs/rescue drugs including but not limited to Epinephrine, norepinephrine, dopamine and dobutamine), such as one compound, such as at least two compounds, such as at least three compounds. When using more than one compound, the compounds may be selected from the same class of compounds, or more preferably the at least two compounds may be selected from different classes of compounds. The compounds may be administered simultaneously or sequentially.

The one or more compounds capable of modulating/preserving the endothelial integrity are particularly useful when administered to human patients in combination with endovascular and/or surgical procedures such as Percutaneous coronary intervention (PCI), PCI with thrombus aspiration, PCI with stents, coronary angiography, Coronary artery bypass surgery (CABG) or thrombolytic therapy. The one or more compounds capable of modulating/preserving the endothelial integrity may be administered in combination with any (classes of) compounds mentioned above (platelet inhibitors, parenteral anticoagulants, Verapamil; adenosine, Sodium nitroprusside, Nitroglycerin, Epinephrine and Beta-blockers), such as one compound, such as at least two compounds, such as at least three compounds when being administered in combination with endovascular and/or surgical procedures such as Percutaneous coronary intervention (PCI), PCI with thrombus aspiration, PCI with stents, coronary angiography, Coronary artery bypass surgery (CABG) or thrombolytic therapy.

When the one or more compounds mentioned above are administered in combination with surgical procedures such as Percutaneous coronary intervention (PCI), PCI with thrombus aspiration, PCI with stents, coronary angiography, Coronary artery bypass surgery (CABG) or thrombolytic therapy the compounds may be administered before, during and/or after the surgery.

Accordingly, in one embodiment one compound is a compound capable of modulating/preserving the endothelium (endothelial modulator) and the at least one other compound is selected from a compound capable of inhibiting the platelets (platelet inhibitor), a parenteral anticoagulant, Verapamil, Adenosine, Sodium nitroprusside, Nitroglycerin, Epinephrine and a Beta-blocker).

In a preferred embodiment a compound capable of modulating/preserving the endothelium and a platelet inhibitor are administered together.

In another preferred embodiment a compound capable of modulating/preserving the endothelium and a platelet inhibitor are administered together in combination with a surgical procedure such as Percutaneous coronary intervention (PCI), PCI with thrombus aspiration, PCI with stents, coronary angiography, Coronary artery bypass surgery (CABG) or thrombolytic therapy. The compounds may be administered before, during and/or after the surgery.

In another preferred embodiment the compound capable of modulating/preserving the endothelial integrity is selected from the group consisting of PGI2, PGX, nitrogen oxide, and prostacyclin or variants thereof is administered together with a platelet inhibitor that is capable of inhibiting the GPIIb/IIIa receptor in combination with a surgical procedure such as Percutaneous coronary intervention (PCI), PCI with thrombus aspiration, PCI with stents, coronary angiography, Coronary artery bypass surgery (CABG) or thrombolytic therapy.

In a specific embodiment of the invention PGI2 and Eptifibade are administered together in combination with a surgical procedure such as Percutaneous coronary intervention (PCI), PCI with thrombus aspiration and PCI with stents.

In one embodiment the invention is in any form performed on human patients, for example patients undergoing any intervention such as Percutaneous coronary intervention (PCI), PCI with thrombus aspiration, PCI with stents, coronary angiography, Coronary artery bypass surgery (CABG) or thrombolytic therapy.

In another embodiment PGI2 and Eptifibade are the only active ingredients.

Combination treatment may include administration of any combination of one or more, such as two or more anti-thrombotic compounds, such as one or more of the following: platelet inhibitors including but not limited to GPIIb/IIIa inhibitors, ADP receptor inhibitors, P2Y1 inhibitors, COX1 and COX2 inhibitors, TX-synthase inhibitors, adenosine uptake inhibitors, GPIb inhibitors, GPVI inhibitors, PAR receptor inhibitors, phosphodiesterase inhibitors, nitroaspirin, albumin conjugated with polyethylene glycol, MP40X, anti-CLEC-2 antibodies, FBP or similar compounds and/or endothelial modulators including but not limited to PGI₂/prostacyclin analogues and variants hereof, prostacyclin/prostacyclin analogue combined with endothelin receptor antagonists, NO, CD39, CD73, compounds involved in redox control, clinical drugs involved in redox control (HMG-CoA reductase inhibitors), Honokiol, compounds modulating S1P-receptors, antibodies and/or other molecules against/antagonizing histones, compounds enhancing/modulating the natural anticoagulant pathways such as the protein C pathway including but not limited to APC, PC, PS, sTM, sEPCR), ATIII pathway (ATIII), TFPI pathway (TFPI), Gβy stimulators and/or any pro-fibrinolytics such as t-PA, u-PA, rt-PA, ru-PA (Actilyse, Metalyse, Rapilysin, Streptase, Urokinase and other compounds containing t-PA and/or rt-PA, uPA, r-uPA and any TAFIa-inhibitors including but not limited to CPU-I, AZD9684, MERGETPA, Compound 21 (UK-396,082) and other compounds with a similar effect.

Thus, in preferred embodiments the compound capable of modulating/preserving the endothelial integrity is selected from the group consisting of PGI2, PGX, nitrogen oxide, CD39, CD73 and prostacyclin or variants thereof, such as beraprost sodium, epoprostenol sodium, iloprost, iloprost in combination with bosentan, iloprost in combination with sildenafil citrate, treprostinil, pegylated treprostinil, treprostinil diethanolamine and treprostinil sodium, 2-{4-[(5,6-diphenylpyrazin-2-yl)(isopropyl)amino]butoxy}-N-(methylsulfonyl)acetamide, {4-[(5,6-diphenylpyrazin-2-yl)(isopropyl)amino]butoxy}acetic acid, 8-[1,4,5-triphenyl-1H-imidazol-2-yl-oxy]octanoic acid, isocarbacyclin, cicaprost, [4-[2-(1,1-Diphenylethylsulfanyl)-ethyl]-3,4-dihydro-2H-benzo[1,4]oxazin-8-yloxy]-acetic acid N-Methyl-d-glucamine, 7,8-dihydro-5-(2-(1-phenyl-1-pyrid-3-yl-methiminoxy)-ethyl)-a-naphthyloxyacetic acid, (5-(2-diphenylmethyl aminocarboxy)-ethyl)-a-naphthyloxyaceticacid, 2-[3-[2-(4,5-diphenyl-2-oxazolyl)ethyl]phenoxy]acetic acid, [3-[4-(4,5-diphenyl-2-oxazolyl)-5-oxazolyl]phenoxy]acetic acid, bosentan, 17[alpha], 20-dimethyl-[DELTA]6,6a-6a-carba PGI1, 15-deoxy-16[alpha]-hydroxy-16[beta], 20-dimethyl-[DELTA]6,6a-6a-carba PGI1 and pentoxifylline (1-{5-oxohexyl}-3,7-dimethylxanthine) and the platelet inhibitor is selected from the group consisting of abciximab, eptifibatide, tirofiban, orbofiban, xemilofiban, lamifiban, XJ757, DUP728 and XR299.

In another equally preferred embodiment the compound capable of modulating/preserving the endothelial integrity is selected from the group consisting of PGI2, PGX, nitrogen oxide, CD39, CD73 and prostacyclin or variants thereof, such as beraprost sodium, epoprostenol sodium, iloprost, iloprost in combination with bosentan, iloprost in combination with sildenafil citrate, treprostinil, pegylated treprostinil, treprostinil diethanolamine and treprostinil sodium, 2-{4-[(5,6-diphenylpyrazin-2-yl)(isopropyl)amino]butoxy}-N-(methylsulfonyl)acetamide, {4-[(5,6-diphenylpyrazin-2-yl)(isopropyl)amino]butoxy}acetic acid, 8-[1,4,5-triphenyl-1H-imidazol-2-yl-oxy]octanoic acid, isocarbacyclin, cicaprost, [4-[2-(1,1-Diphenylethylsulfanyl)-ethyl]-3,4-dihydro-2H-benzo[1,4]oxazin-8-yloxy]-acetic acid N-Methyl-d-glucamine, 7,8-dihydro-5-(2-(1-phenyl-1-pyrid-3-yl-methiminoxy)-ethyl)-a-naphthyloxyacetic acid, (5-(2-diphenylmethyl aminocarboxy)-ethyl)-a-naphthyloxyaceticacid, 2-[3-[2-(4,5-diphenyl-2-oxazolyl)ethyl]phenoxy]acetic acid, [3-[4-(4,5-diphenyl-2-oxazolyl)-5-oxazolyl]phenoxy]acetic acid, bosentan, 17[alpha], 20-dimethyl-[DELTA]6,6a-6a-carba PGI1, 15-deoxy-16[alpha]-hydroxy-16[beta], 20-dimethyl-[DELTA]6,6a-6a-carba PGI1 and pentoxifylline (1-{5-oxohexyl}-3,7-dimethylxanthine) and the platelet inhibitor is selected from the group consisting of AR-C69931MX, Ticlopidine, Clopidogrel, Prasugrel, AZD6140 and cangrelor, ticagrelor.

In other preferred embodiments the platelet inhibitor is capable of inhibiting the GPIIb/IIIa receptor and has a half time of less than 3 hours (such as eptifibatide), preferably less than 2.5 hours (such as tirofiban) and the compound capable of modulating/preserving the endothelial integrity a half time of less than has a half time of less than 4 hours (such as Treprostinil), preferably less than 1 hours (such as Beraprost (35-40 min)), more preferably less than ½ hour (such as Iloprost (20-30 min)), even more preferably less than 5 min (such as PGI2 (flolan) or prostacyclin (Epoprostenol) (0, 5-3 min)). In a less preferred embodiment the platelet inhibitor is capable of inhibiting the GPIIb/IIIa receptor is Abciximab.

In other preferred embodiments the platelet inhibitor is capable of inhibiting platelet ADP receptor P2Y12 and has a half time of 12 hours (such as Ticlopidine or pradugrel), preferably less than 8 hours (such as Clopidogrel), more preferably about 3-5 min (such as cangrelor) and the compound capable of modulating/preserving the endothelial integrity a half time of less than has a half time of less than 4 hours (such as Treprostinil), preferably less than 1 hours (such as Beraprost (35-40 min)), more preferably less than ½ hour (such as Iloprost (20-30 min)), even more preferably less than 5 min (such as PGI2 (flolan) or prostacyclin (Epoprostenol) (0, 5-3 min)).

In particular a combination of platelet inhibitors and compound capable of modulating/preserving endothelial integrity is envisaged by the present invention, such as a combination of a prostacyclin and a GPIIb/IIIa platelet inhibitor, optionally further combined with other compounds or with surgical procedures as described herein above.

The compounds to be applied in the method of the present invention may be administered with at least one other compound. The compounds may be administered simultaneously, either as separate formulations or combined in a unit dosage form, or administered sequentially.

### Dosages

As used herein, “dose” shall mean any concentration of the agents administered to the patient resulting in inhibition of the aggregating/clot forming properties of the platelets and/or maintaining the endothelium in a quiescent state and/or a reduced resistance of the thrombus to fibrinolysis and/or preserving the platelet count and/or function. A dose sufficient to produce the desired effect in relation to the conditions for which it is administered shall be described as the “effective dose” or “effective amount”.

As will be understood by the person skilled in the art, amounts effective for this purpose will depend on the number and functionality of circulating platelets and endothelial cells in the patient and the number of receptors on the respective platelets and endothelial cells.

The dosage requirements will vary with the particular drug composition employed, the route of administration and the particular subject being treated. Ideally, a patient to be treated by the present method will receive a pharmaceutically effective amount of the compound in the maximum tolerated dose, generally no higher than that required before drug resistance develops.

Administration of the compounds and/or compositions of the present invention are to be given to a subject resulting in a systemic concentration of the compounds. Methods of administration include enteral, such as oral, sublingual, gastric or rectal and/or parenterally, that is by intravenous, intraarterial, intramuscular, subcutaneous, intranasal, intrapulmonary, intrarectal, intravaginal or intraperitoneal administration. The subcutaneous and intravenous forms of parenteral administration are generally preferred. Appropriate dosage forms for such administration may be prepared by conventional techniques. The compounds may also be administered by inhalation that is by intranasal and oral inhalation administration. Appropriate dosage forms for such administration, such as an aerosol formulation or a metered dose inhaler, may be prepared by conventional techniques.

The compounds according to the invention may be administered with at least one other compound. The compounds may be administered simultaneously, either as separate formulations or combined in a unit dosage form, or administered sequentially.

Normally the dose should be capable of preventing or lessening the severity or spread of the condition or indication being treated. The exact dose will depend on the circumstances, such as the condition being treated, the administration schedule, whether the compounds are administered alone or in conjunction with another therapeutic agent, the plasma half-life of the compounds and the general health of the subject.

The compounds disclosed herein are generally well known to a person skilled in the art and the appropriate dosages for their use are disclosed in pharmacopeias, pharmaceutical handbooks, and patient information leaflets. Thus the compounds of the present invention may be administered n the dosages recommended by the manufacturers or as are known to be efficient to those skilled in the art, i.e. medical practitioners.

As will be understood by the person skilled in the art, amounts effective for this purpose will depend on the severity of the disease or injury as well as the weight and general state of the subject. The dose is preferably given by the parenteral administration route, notably the intravenous, intraarterial, intramuscular and/or the subcutaneous, sublingual, trans-mucosal, intrapulmonal and intra-alveolar route.

The dosages given in the following is contemplated to be in the same order of magnitude irrespective of the parenteral administration route.

For all methods of use disclosed herein for the compounds, the daily parenteral dosage regimen about 0.001 to about 80 mg/kg of total body weight. The daily oral dosage regimen will preferably be from about 0.01 to about 80 mg/kg of total body weight. The daily topical dosage regimen will preferably be from 0.1 mg to 150 mg, administered one to four, preferably two or three times daily. The daily inhalation dosage regimen will preferably be from about 0.01 mg/kg to about 1 mg/kg per day. It will also be recognized by one of skill in the art that the optimal quantity and spacing of individual dosages of a compound or a pharmaceutically acceptable salt thereof will be determined by the nature and extent of the condition being treated, the form, route and site of administration, and the particular patient being treated, and that such optimums can be determined by conventional techniques. It will also be appreciated by one of skill in the art that the optimal course of treatment, i.e., the number of doses of a compound or a pharmaceutically acceptable salt thereof given per day for a defined number of days, can be ascertained by those skilled in the art using conventional course of treatment determination tests.

The term “unit dosage form” as used herein refers to physically discrete units suitable as unitary dosages for human and animal subjects, each unit containing a predetermined quantity of a compound, alone or in combination with other agents, calculated in an amount sufficient to produce the desired effect in association with a pharmaceutically acceptable diluent, carrier, or vehicle. The specifications for the unit dosage forms of the present invention depend on the particular compound or compounds employed and the effect to be achieved, as well as the pharmacodynamics associated with each compound in the host

It is an object of the present invention that the compounds and/or compositions herein disclosed are administered systemically. It is also an object of the present invention that the compounds are administered parenterally, preferably intravenously and/or intrarterially.

### Pharmaceutical Compositions of the Invention and its Use

The present invention also relates to a pharmaceutical composition comprising any combination of any of the compounds mentioned above (endothelial modulators, platelet inhibitors, GPIIb/IIIa receptor antagonists), such as one compound, such as at least two compounds, such as at least three compounds and one or more pharmaceutically acceptable carriers or excipients. Such pharmaceutically acceptable carrier or excipient as well as suitable pharmaceutical formulation methods are well known in the art (see for example Remington's Pharmaceutical Sciences, 18th ed., Mack Publishing Company, Easton, Pa. (1990). In a preferred embodiment the platelet inhibiting/endothelial protecting variants are prepared in a parenteral composition. Such methods for preparing parenterally administrable compositions will also be known or apparent to those skilled in the art and are described in more detail in, for example, Remington's Pharmaceutical Sciences, 18th ed., Mack Publishing Company, Easton, Pa. (1990). As used herein, the term “pharmaceutical acceptable” means carriers or excipients that does not cause any untoward effects in subjects to whom it is administered.

The compositions for parenteral administration comprise the platelet antiaggregatory agents of the invention in combination with, preferably dissolved in, a pharmaceutically acceptable carrier, preferably an aqueous carrier. A variety of aqueous carriers may be used, such as water, buffered water, saline e.g. such as 0.7%, 0.8%, 0.9% or 1%, glycine such as 0.2%, 0.3%, 0.4% or 0.5% and the like. Normally, it is aimed that the composition has an osmotic pressure corresponding to a 0.9% w/w sodium chloride solution in water. Moreover, as known by a person skilled in the art, dependent on the specific administration route, pH may be adjusted within suitable ranges centred around pH 7.4. The compositions may be sterilised by conventional, well-known sterilisation techniques. The resulting aqueous solutions may be packaged for use or filtered under aseptic conditions and lyophilised, the lyophilised preparation being combined with a sterile aqueous solution prior to administration.

The compositions may contain pharmaceutically acceptable auxiliary substances as required to approximate physiological conditions, such as pH adjusting and buffering agents, stabilizing agents, preservatives, non-ionic surfactants or detergents, antioxidants, tonicity adjusting agents and the like, for example, sodium acetate, sodium lactate, sodium chloride, potassium chloride, calcium chloride, etc.

The main routes of drug delivery, in the treatment method are intravenous, oral, and topical, as will be described below. Other drug-administration methods, such as subcutaneous injection or via inhalation, which are effective to deliver the drug to a target site or to introduce the drug into the bloodstream, are also contemplated.

Compounds of the invention may be administered parenterally, that is by intravenous, intramuscular, subcutaneous intranasal, intrarectal, intravaginal or intraperitoneal administration. Appropriate dosage forms for such administration may be prepared by conventional techniques. The compounds may also be administered by inhalation that is by intranasal and oral inhalation administration. Appropriate dosage forms for such administration, such as an aerosol formulation or a metered dose inhaler, may be prepared by conventional techniques.

The compounds are preferably administered intravenously and/or intraalveolar and it may be administered by continuous or pulsatile infusion or as a bolus.

The compounds to be applied in the method of the present invention may be administered with at least one other compound. The compounds may be administered simultaneously, either as separate formulations or combined in a unit dosage form, or administered sequentially. It is thus also contemplated that one compound may be administered intravenously for example in combination with another compound that is administered orally.

### Clinical Indications

As described herein above the present invention relates to treatment and/or prevention of ischemic events in patients with cardiovascular disease. The present invention is particularly useful for patients having or being at increased risk of development of an ischemic event such as an acute myocardial infarction and/or no-reflow phenomena and/or ischemia-reperfusion injury.

The present invention thus in one embodiment relates to treatment and/or prevention ischemic events in patients with Acute coronary syndrome (ACS).

ACS is a set of signs and symptoms (syndrome) related to the heart. ACS is compatible with a diagnosis of acute myocardial ischemia, but it is not characteristic of the diagnosis.

The sub-types of acute coronary syndrome include unstable angina (UA, not associated with heart muscle damage), and two forms of myocardial infarction (MI, heart attack), in which heart muscle is damaged. These types are named according to the appearance of the electrocardiogram (ECG/EKG) as non-ST segment elevation myocardial infarction (NSTEMI) and ST segment elevation myocardial infarction (STEMI).

ACS should be distinguished from stable angina, which develops during exertion and resolves at rest. In contrast with stable angina, unstable angina occurs suddenly, often at rest or with minimal exertion, or at lesser degrees of exertion than the individual's previous angina (“crescendo angina”). New onset angina is also considered unstable angina, since it suggests a new problem in a coronary artery.

Though ACS is usually associated with coronary thrombosis, it can also be associated with cocaine use. Cardiac chest pain can also be precipitated by anemia, bradycardias (excessively slow heart rate) or tachycardias (excessively fast heart rate).

In another aspect the present invention relates to treatment and/or prevention of ischemic events in cardiovascular patients before, during and/or after surgical procedures such as PCI, coronary angiography, Coronary artery bypass surgery (CABG) or thrombolytic therapy.

Patients about to undergo or having undergone surgical procedures may be patients with any cardiovascular disease or preferably patients with ACS.

Accordingly, in one embodiment the present invention relates to a method of treating a patient with cardiovascular disease such as ACS, wherein said patient is at increased risk of development of an ischemic event such as an acute myocardial infarction and/or no-reflow phenomena and/or ischemia-reperfusion injury by administering one or more compounds as discussed above belonging to one or more of the classes:
1. Platelet inhibitors (described herein above in detail including Aspirin (ASA), Thienopyridins, GPIIb/IIIa inhibitors);
2. Parenteral anticoagulants (unfractioned heparin (UFH), bivalirudin, enoxaparin, and fondaparinux);
3. Verapamil;
4. Adenosine;
5. Sodium nitroprusside;
6. Nitroglycerin;
7. Pressor drugs/rescue drugs including but not limited to Epinephrine, norepinephrine, dopamine and dobutamin
8. Beta-blockers;
   or in combination with surgical methods such as Percutaneous coronary intervention (PCI), PCI with thrombus aspiration, PCI with stents, coronary angiography, Coronary artery bypass surgery (CABG) or thrombolytic therapy.

Optionally combined with further compounds.

Another aspect of the invention relates to purpura fulminans. Purpura fulminans is a haemorrhagic condition usually associated with sepsis or previous infection. It is a hemorrhagic infarction of the skin that is rapidly progressive and is accompanied by vascular collapse and disseminated intravascular coagulation. There are three forms of the disease classified by the triggering mechanisms: Neonatal purpura fulminans, which occurs mainly in babies and small children; Idiopathic (or chronic) purpura fulminans which follows a bacterial or viral illness and occurs after a variable latent period; and Acute infectious purpura fulminans, which is the most common form of purpura fulminans and occurs superimposed on a bacterial infection. Administration of the compounds described herein may also be useful in the treatment and/or prevention of purpura fulminans.

Therefore one aspect of the invention relates to a pharmaceutical composition comprising one or more, such as two or more compounds capable of modulating/preserving the endothelial integrity and one or more platelet inhibitors for use in the treatment and/or prevention of purpura fulminans.

Thus, in an embodiment the compound capable of modulating/preserving the endothelial integrity is selected from the group consisting of PGI2, PGX, nitrogen oxide, and prostacyclin or variants thereof is administered together with a platelet inhibitor that is capable of inhibiting the GPIIb/IIIa receptor for the treatment and/or prevention of purpura fulminans.

In a specific embodiment of the invention PGI2 and Eptifibade are administered together for the treatment and/or prevention of purpura fulminans.

A further aspect of the present invention other ischemic indications are of relevance, these include frostbite, wounds, ulcers and sore healing, the ischemic, and especially the ischemia-reperfusion injuries or events that follow the removal of pulmonary emboli, the removal of intra cerebral venous and/or arterial thrombi and/or emboli, and the removal of gastrointestinal thromboses and/or emboli.

Any combination of compounds mention herein may be administered to treat and or prevent frostbite. Frostbite occurs when, due to cold temperatures, blood vessels close to the skin start to constrict, and blood is shunted away from the extremities via the action of glomus bodies. The same response may also be a result of exposure to high winds. This constriction helps to preserve core body temperature. In extreme cold, or when the body is exposed to cold for long periods, this protective strategy can reduce blood flow in some areas of the body to dangerously low levels. This lack of blood leads to the eventual freezing and death of skin tissue in the affected areas. All degrees of frostbite are treatable with the present combination of compounds including chilblains, frostnip, blisters and frostbite of varying depths.

Therefore one aspect of the invention relates to a pharmaceutical composition comprising one or more, such as two or more compounds capable of modulating/preserving the endothelial integrity and one or more platelet inhibitors for use in the treatment and/or prevention of frostbite.

Thus in an embodiment the compound capable of modulating/preserving the endothelial integrity is selected from the group consisting of PGI2, PGX, nitrogen oxide, and prostacyclin or variants thereof is administered together with a platelet inhibitor that is capable of inhibiting the GPIIb/IIIa receptor for the treatment and/or prevention of frostbite.

In a specific embodiment of the invention PGI2 and Eptifibade are administered together for the treatment and/or prevention of frostbite.

The wounds from which ulcers arise can be caused by a wide variety of factors, but the main cause is impaired blood circulation. Especially, chronic wounds and ulcers are caused by poor circulation, either through cardiovascular issues or external pressure from a bed or a wheelchair. The healing of wounds, ulcers, sores and the like is thus an indication that may be treated according to the present invention by the administration of a combination of compounds as cited herein.

Therefore one aspect of the invention relates to a pharmaceutical composition comprising one or more, such as two or more compounds capable of modulating/preserving the endothelial integrity and one or more platelet inhibitors for use in the treatment of ulcers, sores and wounds.

Thus in an embodiment the compound capable of modulating/preserving the endothelial integrity is selected from the group consisting of PGI2, PGX, nitrogen oxide, and prostacyclin or variants thereof is administered together with a platelet inhibitor that is capable of inhibiting the GPIIb/IIIa receptor for the treatment of ulcers, sores and wounds and assists in the healing hereof.

In a specific embodiment of the invention PGI2 and Eptifibade are administered together for the treatment of ulcers, sores and wounds and assists in the healing hereof.

The blockage of a capillary bed by an embolus or thrombus results in an ischemic condition and the removal of the embolus or thrombus is likely to give rise to ischemic reperfusion injuries. It is an object of the present invention to treat and/or prevent the ischemic injuries and/or the ischemia-reperfusion injuries or events that follow the removal of any of the following conditions: pulmonary emboli, intra cerebral venous and/or arterial thrombi and/or emboli, and gastrointestinal thromboses and/or emboli. Any combination of compounds herein mentioned may be used for said treatment and/or prevention of the ischemic and/or ischemic reperfusion injuries that are associated with the presence of or removal of pulmonary emboli, intra cerebral venous and/or arterial thrombi and/or emboli, and gastrointestinal thromboses and/or emboli.

Therefore one aspect of the invention relates to a pharmaceutical composition comprising one or more, such as two or more compounds capable of modulating/preserving the endothelial integrity and one or more platelet inhibitors for the treatment and/or prevention of the ischemic and/or ischemic reperfusion injuries that are associated with the presence of or removal of pulmonary emboli, intra cerebral venous and/or arterial thrombi and/or emboli, and gastrointestinal thromboses and/or emboli.

Thus in an embodiment the compound capable of modulating/preserving the endothelial integrity is selected from the group consisting of PGI2, PGX, nitrogen oxide, and prostacyclin or variants thereof is administered together with a platelet inhibitor that is capable of inhibiting the GPIIb/IIIa receptor for the treatment and/or prevention of the ischemic and/or ischemic reperfusion injuries that are associated with the presence of or removal of pulmonary emboli, intra cerebral venous and/or arterial thrombi and/or emboli, and gastrointestinal thromboses and/or emboli.

In a specific embodiment of the invention PGI2 and Eptifibade are administered together for the treatment and/or prevention of the ischemic and/or ischemic reperfusion injuries that are associated with the presence of or removal of pulmonary emboli, intra cerebral venous and/or arterial thrombi and/or emboli, and gastrointestinal thromboses and/or emboli.

Another aspect of the present invention relates to the use of the herein disclosed compounds and combinations hereof for the treatment of Melioidosis. Melioidosis is an infectious disease caused by a Gram-negative bacterium, Burkholderia pseudomallei, found in soil and water. It exists in acute and chronic forms. Symptoms may include pain in chest, bones, or joints; cough; skin infections, lung nodules and pneumonia.

Therefore one aspect of the invention relates to a pharmaceutical composition comprising one or more, such as two or more compounds capable of modulating/preserving the endothelial integrity and one or more platelet inhibitors for use in the treatment of Melioidosis.

Thus in an embodiment the compound capable of modulating/preserving the endothelial integrity is selected from the group consisting of PGI2, PGX, nitrogen oxide, and prostacyclin or variants thereof is administered together with a platelet inhibitor that is capable of inhibiting the GPIIb/IIIa receptor for the treatment of Melioidosis.

In a specific embodiment of the invention PGI2 and Eptifibade are administered together for the treatment of Melioidosis.

### Embodiments

In one embodiment the invention relates to a pharmaceutical composition comprising one or more, such as two or more compounds capable of modulating/preserving the endothelial integrity and for use in the treatment and/or prevention of ischemic events in patients being at increased risk of developing an ischemic event such as an acute myocardial infarction and/or no-reflow phenomena and/or ischemia-reperfusion injury.

In another embodiment the above composition further comprises one or more platelet inhibitors.

In one embodiment, the invention thus relates to a composition wherein the compound capable of modulating/preserving the endothelial integrity is selected from the group consisting of CD39 and CD73.

In one embodiment, the invention thus relates to a composition wherein the compound capable of modulating/preserving the endothelial integrity is a compound involved in redox control of endothelial functions.

In one embodiment, the invention thus relates to a composition wherein the compound capable of modulating/preserving the endothelial integrity is selected from the group consisting of L-Arginine and tetrahydrobiopterin, Antioxidants (Ascorbate, Glutathione, α-tocopherol, ubiquinol-10, Probucol), Iron chelators, Polyphenols

In one embodiment, the invention thus relates to a composition wherein the compound capable of modulating/preserving the endothelial integrity is selected from the group consisting of HMG-CoA reductase inhibitors (Fluvastatin, Lovastatin, Pravastatin, Simvastatin), Angiotensin-receptor antagonists and ACE inhibitors (Captopril, Zofenopril, Enalapril, Ramipril, Quinapril, Perindopril, Lisinopril, Benazepril, Fosinopril, Casokinins, lactokinins), Peroxisome proliferator-activated receptors (PPARs), NADPH oxidase, Xanthine oxidase, PETN, Heparan sulfates (PI-88), heparan sulfate mimetics, Activators of oxidized/heme-free sGC (BAY 58-2667), Anti-PECAM/SOD.

In one embodiment, the invention thus relates to a composition wherein the compound capable of modulating/preserving the endothelial integrity is Honokiol.

In one embodiment, the invention thus relates to a composition wherein the compound capable of modulating/preserving the endothelial integrity is a compound that directly modulates endothelial barrier function through modulating effects on sphingosine-1-phosphate (S1P)-receptors.

In one embodiment, the invention thus relates to a composition wherein the compound capable of modulating/preserving the endothelial integrity is selected from the group consisting of TY720, AA-R, AAL-S, KRP-203, AUY954, CYM-5442, SEW2871, W146, W140, VPC44116, VPC23019, JTE-013).

In one embodiment, the invention thus relates to a composition wherein the compound capable of modulating/preserving the endothelial integrity is an antibody and/or another molecule against/antagonizing histones through their inhibition histone-mediated endothelial damage and/or microthrombi formation and/or fibrin deposition.

In one embodiment, the invention thus relates to a composition wherein the compound capable of modulating/preserving the endothelial integrity is a compound enhancing the natural anticoagulant pathways and hence protecting the endothelium such as but not exclusively: Protein C pathway (Activated protein C (APC, Drotrecogin alfa), protein C, compounds that either mimics and/or protects from degradation and/or enhances soluble thrombomodulin and/or EPCR and/or protein S), Antithrombin III (ATIII) (or ATIII like compounds and/or compounds that enhance ATIII function) and tissue factor pathway inhibitor (TFPI) (or TFPI compounds and/or compounds that enhance TFPI function).

In one embodiment, the invention thus relates to a composition wherein the compound capable of modulating/preserving the endothelial integrity is a compound that maintain and/or promote Gβy function and/or signalling following endothelial PAR activation to ensure reannealing of adherens junctions opened following inflammatory PAR mediated activation of Gα

In one embodiment, the invention thus relates to a composition wherein the compound capable of modulating/preserving the endothelial integrity is Defibrotide.

In one embodiment, the invention thus relates to a composition wherein the platelet inhibitor has a half time of less than 3 hours (such as eptifibatide), preferably less than 2.5 hours (such as tirofiban), more preferably less than 1 hour.

In one embodiment, the invention thus relates to a composition wherein the platelet inhibitor is capable of inhibiting platelet ADP receptor P2Y12.

In one embodiment, the invention thus relates to a composition wherein the platelet inhibitor is selected from the group consisting of AR-C69931 MX, Ticlopidine, Clopidogrel, Prasugrel, AZD6140 and cangrelor, ticagrelor.

In one embodiment, the invention thus relates to a composition wherein the platelet inhibitor has a half time of less than 12 hours (such as Ticlopidine), preferably less than 8 hours (such as Clopidogrel), more preferably about 3-5 min (such as cangrelor).

In one embodiment of the invention the platelet inhibitor is capable of inhibiting platelet receptor P2Y1.

In one embodiment of the invention the platelet inhibitor is selected from the group consisting of MRS2500, MRS2298, MRS2496, A2P5P, A3P5P, ATP, 2-MeSATP, and 2-CIATP.

In one embodiment of the invention the platelet inhibitor is a haemoglobin conjugated with polyethylene glycol, a compound that besides its platelet inhibitory function also improves oxygenation of the microvasculature, such as but not exclusively MP40X (Hemospan, polyethylene glycol-hemoglobin complexes)

In one embodiment of the invention the platelet inhibitor is an antibody and/or inhibitors of C-type lectin-like receptor 2 (CLEC-2).

In one embodiment of the invention the platelet inhibitor is a high-energy glycolitic metabolites like fructose-1,6-bisphosphate (FBP).

In one embodiment of the invention the platelet inhibitor is capable of inhibiting the GPIIb/IIIa receptor and has a half time of less than 3 hours (such as eptifibatide), preferably less than 2.5 hours (such as tirofiban), more preferably less than 1 hour, and the compound capable of modulating/preserving the endothelial integrity a half time of less than has a half time of less than 4 hours (such as Treprostinil), preferably less than 1 hours (such as Beraprost (35-40 min)), more preferably less than ½ hour (such as Iloprost (20-30 min)), even more preferably less than 5 min (such as PGI2 (flolan) or prostacyclin (Epoprostenol) (0, 5-3 min)).

In one embodiment of the invention the platelet inhibitor is capable of inhibiting platelet ADP receptor P2Y12 and has a half time of 12 hours (such as Ticlopidine), preferably less than 8 hours (such as Clopidogrel), more preferably about 3-5 min (such as cangrelor and the compound capable of modulating/preserving the endothelial integrity a half time of less than has a half time of less than 4 hours (such as Treprostinil), preferably less than 1 hours (such as Beraprost (35-40 min)), more preferably less than ½ hour (such as Iloprost (20-30 min even more preferably less than 5 min (such as PGI2 (flolan) or prostacyclin (Epoprostenol) (0, 5-3 min)).

In one embodiment of the invention the platelet inhibitor is selected from the group consisting of AR-C69931 MX, Ticlopidine, Clopidogrel, Prasugrel, AZD6140 and cangrelor, ticagrelor and the compound capable of modulating/preserving the endothelial integrity is selected from the group consisting of PGI2, PGX, nitrogen oxide, CD39, CD73 and prostacyclin or variants thereof, such as beraprost sodium, epoprostenol sodium, iloprost, iloprost in combination with bosentan, iloprost in combination with sildenafil citrate, treprostinil, pegylated treprostinil, treprostinil diethanolamine and treprostinil sodium, 2-{4-[(5,6-diphenylpyrazin-2-yl)(isopropyl)amino]butoxy}-N-(methylsulfonyl)acetamide, {4-[(5,6-diphenylpyrazin-2-yl)(isopropyl)amino]butoxy}acetic acid, 8-[1,4,5-triphenyl-1H-imidazol-2-yl-oxy]octanoic acid, isocarbacyclin, cicaprost, [4-[2-(1,1-Diphenylethylsulfanyl)-ethyl]-3,4-dihydro-2H-benzo[1,4]oxazin-8-yloxy]-acetic acid N-Methyl-d-glucamine, 7,8-dihydro-5-(2-(1-phenyl-1-pyrid-3-yl-methiminoxy)-ethyl)-a-naphthyloxyacetic acid, (5-(2-diphenylmethyl aminocarboxy)-ethyl)-a-naphthyloxyaceticacid, 2-[3-[2-(4,5-diphenyl-2-oxazolyl)ethyl]phenoxy]acetic acid, [3-[4-(4,5-diphenyl-2-oxazolyl)-5-oxazolyl]phenoxy]acetic acid, bosentan, 17[alpha], 20-dimethyl-[DELTA]6,6a-6a-carba PGI1, 15-deoxy-16[alpha]-hydroxy-16[beta], 20-dimethyl-[DELTA]6,6a-6a-carba PGI1 and pentoxifylline (1-{5-oxohexyl}-3,7-dimethylxanthine).

In one embodiment of the invention the platelet inhibitor is selected from the group consisting of abciximab, eptifibatide, tirofiban, orbofiban, xemilofiban, lamifiban, XJ757, DUP728 and XR299 and the compound capable of modulating/preserving the endothelial integrity is selected from the group consisting of PGI2, PGX, nitrogen oxide, CD39, CD73 and prostacyclin or variants thereof, such as beraprost sodium, epoprostenol sodium, iloprost, iloprost in combination with bosentan, iloprost in combination with sildenafil citrate, treprostinil, pegylated treprostinil, treprostinil diethanolamine and treprostinil sodium, 2-{4-[(5,6-diphenylpyrazin-2-yl)(isopropyl)amino]butoxy}-N-(methylsulfonyl)acetamide, {4-[(5,6-diphenylpyrazin-2-yl)(isopropyl)amino]butoxy}acetic acid, 8-[1,4,5-triphenyl-1H-imidazol-2-yl-oxy]octanoic acid, isocarbacyclin, cicaprost, [4-[2-(1,1-Diphenylethylsulfanyl)-ethyl]-3,4-dihydro-2H-benzo[1,4]oxazin-8-yloxy]-acetic acid N-Methyl-d-glucamine, 7,8-dihydro-5-(2-(1-phenyl-1-pyrid-3-yl-methiminoxy)-ethyl)-a-naphthyloxyacetic acid, (5-(2-diphenylmethyl aminocarboxy)-ethyl)-a-naphthyloxyaceticacid, 2-[3-[2-(4,5-diphenyl-2-oxazolyl)ethyl]phenoxy]acetic acid, [3-[4-(4,5-diphenyl-2-oxazolyl)-5-oxazolyl]phenoxy]acetic acid, bosentan, 17[alpha], 20-dimethyl-[DELTA]6,6a-6a-carba PGI1, 15-deoxy-16[alpha]-hydroxy-16[beta], 20-dimethyl-[DELTA]6,6a-6a-carba PGI1 and pentoxifylline (1-{5-oxohexyl}-3,7-dimethylxanthine).

In one embodiment, the invention thus relates to a composition, wherein the platelet inhibitor is capable of inhibiting the platelet COX1 and/or COX2 pathways such as salicylates, arylalkanoic acids, 2-Arylpropionic acids, N-Arylanthranilic acids, pyrazolidine derivatives and oxicams.

In one embodiment, the invention thus relates to a composition wherein the platelet inhibitor is capable of inhibiting Thromboxane-synthase, such as Flavonoids, such as Apigenin, and TP-antagonists such as SQ29548, Bay u 3405, BM 13.177.

In one embodiment, the invention thus relates to a composition wherein the platelet inhibitor is capable of inhibiting adenosine uptake in the platelets such as dipyramidol such as Persantin, Asasantin, Aggrenox.

In one embodiment, the invention thus relates to a composition wherein the platelet inhibitor is capable of inhibiting the platelet GPIb receptor, such as mAB lb-23, mAB 6B4, R9alpha557 peptide, aurintricarboxylic acid (ATA), crotalin, agkistin, peptide (Trp-Ile-Arg-Arg-Pro-Phe-Phe-Pro-Phe) from alpha B-crystallin

In one embodiment, the invention thus relates to a composition wherein the platelet inhibitor is capable of inhibiting the platelet GPVI receptor, such as EXP3179, triplatin-1 and -2, JAQ1, mAB 10B12, mAB 1C3, mAb 12G1.

In one embodiment, the invention thus relates to a composition wherein the platelet inhibitor is capable of inhibiting the platelet PAR receptors, such as thrombin inhibitors, heterocycle-based peptide-miimetic antagonists of PAR-1, RWJ-56110 and RWJ-58259, SCH 79797 SCH 203099 and PAR4 antagonists such as trans-cinnamoyl-YPGKF-amide (tc-Y—NH(2)) and palmitoyl-SGRRYGHALR-amide (P4pal10), PAR-2 antagonist ENMD-1068, PAR2 monoclonal antibody SAM-11.

In one embodiment, the invention thus relates to a composition wherein the platelet inhibitor is Phosphodiesterase inhibitor PDE3 such as Cilostazol.

In one embodiment, the invention thus relates to a composition wherein the platelet inhibitor is Nitroaspirin (NCX4016).

In one embodiment, the invention thus relates to a composition wherein the platelet inhibitor is a Polyethylene Glycol-Conjugated Albumin.

### DETAILED DESCRIPTION OF DRAWINGS

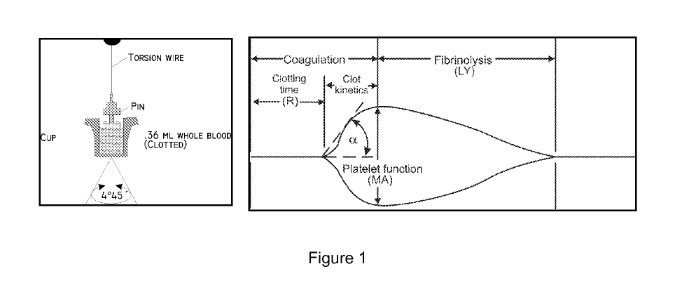

FIG. 1: Recording haemostatic activity using TEG assay.

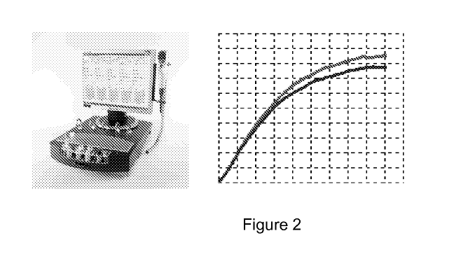

FIG. 2: MultiPlate continuously records platelet aggregation. The increase of impedance by the attachment of platelets onto the Multiplate sensors is transformed to arbitrary aggregation units (AU) and plotted against time.

### EXAMPLES

### Example 1

In an upcoming open label, randomized, single centre study the safety of 24 h 0.5 ng/min/kg continuous dosing of Flolan® (prostacyclin) in patients undergoing pPCI due to STEMI, in addition to standard treatment with Integrillin® (GPIIB/IIIA inhibitor) according to local practice. Inclusion and randomisation of patients will occur 23 hours after the PCI procedure.

Five hours after the standard Integrillin® treatment for pPCI patients (2.0 μg/kg/min i.v infusion for 18 hours) will be stopped, it will be started again at lower dose Integrilin® (0.5 μg/kg/min, 25% of standard dose) in combination with either 0.5 ng/kg/min Flolan® (active) or saline (placebo) for 24 hours. During the study blood samples will be taken at different timepoints (pre-Integrillin® infusion, pre-commencing infusion with study drug (low dose Integrillin® with Flolan or Saline (placebo) after 1 h, 6 h, 12 h, 24 h and 48 h), these blood samples are safety bloodsample to evaluate biochemistry and haematology as well as for efficacy parameters, endothelial markers (sE-selectin (early marker), ICAM-1 (late marker), HMGB1 (necrosis marker), vWF, sTM and sCD40L) and platelet aggregation and clot strength as evaluated by Multiplate and TEG.

Patients will be observed and assessed continuously with regards to bleeding, according to the TIMI and GUSTO bleeding criteria. ECGs will be taken as per normal procedures for PCI patients 24 h post PCI and 48 h post PCI. Patients will participate in the study for two days.

### REFERENCES

A randomised, blinded, trial of clopidogrel versus aspirin in patients at risk of ischaemic events (CAPRIE). CAPRIE Steering Committee. Lancet 1996; 348:1329-1339
Adams V, Linke A, Krankel N et al. Impact of regular physical activity on the NAD(P)H oxidase and angiotensin receptor system in patients with coronary artery disease. Circulation 2005; 111:555-562
Bahlmann F H, de G K, Mueller O et al. Stimulation of endothelial progenitor cells: a new putative therapeutic effect of angiotensin II receptor antagonists. Hypertension 2005; 45:526-529
Bihari D J, Tinker J. The therapeutic value of vasodilator prostaglandins in multiple organ failure associated with sepsis. Intensive Care Med 1988; 15:2-7
Charakida M, Donald A E, Terese M et al. Endothelial dysfunction in childhood infection. Circulation 2005; 111:1660-1665
Cines D B, Pollak E S, Buck C A et al. Endothelial cells in physiology and in the pathophysiology of vascular disorders. Blood 1998; 91:3527-3561
De Meyer G R, Martinet W. Autophagy in the cardiovascular system. Biochim Biophys Acta 2009; 1793:1485-1495
Goepfert C, Imai M, Brouard S et al. CD39 modulates endothelial cell activation and apoptosis. Mol Med 2000; 6:591-603
Grande P O, Moller A D, Nordstrom C H et al. Low-dose prostacyclin in treatment of severe brain trauma evaluated with microdialysis and jugular bulb oxygen measurements. Acta Anaesthesiol Scand 2000; 44:886-894
Hambrecht R, Wolf A, Gielen S et al. Effect of exercise on coronary endothelial function in patients with coronary artery disease. N Engl J Med 2000; 342:454-460
Hansson G K. Inflammation, atherosclerosis, and coronary artery disease. N Engl J Med 2005; 352:1685-1695
Hingorani A D, Cross J, Kharbanda R K et al. Acute systemic inflammation impairs endothelium-dependent dilatation in humans. Circulation 2000; 102:994-999
Hornig B, Drexler H. Reversal of endothelial dysfunction in humans. Coron Artery Dis 2001; 12:463-473
Knezevic N, Tauseef M, Thennes T et al. The G protein {beta}{gamma} subunit mediates reannealing of adherens junctions to reverse endothelial permeability increase by thrombin. J Exp Med 2009
Landmesser U, Bahlmann F, Mueller M et al. Simvastatin versus ezetimibe: pleiotropic and lipid-lowering effects on endothelial function in humans. Circulation 2005; 111:2356-2363
Landmesser U, Hornig B, Drexler H. Endothelial function: a critical determinant in atherosclerosis? Circulation 2004; 109:1127-1133
Laufs U, Bohm M. Cardiac effects of statins—advancements and open questions. Cardiovasc Res 2005; 66:427-429
Marsolais D, Rosen H. Chemical modulators of sphingosine-1-phosphate receptors as barrier-oriented therapeutic molecules. Nat Rev Drug Discov 2009; 8:297-307
Morabito F, Gentile M, Gay F et al. Insights into defibrotide: an updated review. Expert Opin Biol Ther 2009; 9:763-772
Naredi S, Olivecrona M, Lindgren C et al. An outcome study of severe traumatic head injury using the “Lund therapy” with low-dose prostacyclin. Acta Anaesthesiol Scand 2001; 45:402-406
Patti G, Pasceri V, Melfi R et al. Impaired flow-mediated dilation and risk of restenosis in patients undergoing coronary stent implantation. Circulation 2005; 111:70-75
Scheeren T, Radermacher P. Prostacyclin (PGI2): new aspects of an old substance in the treatment of critically ill patients. Intensive Care Med 1997; 23:146-158
Shimokawa H, Yasuda S. Myocardial ischemia: Current concepts and future perspectives. J Cardiol 2008; 52:67-78
Solomon D H, Karlson E W, Rimm E B et al. Cardiovascular morbidity and mortality in women diagnosed with rheumatoid arthritis. Circulation 2003; 107:1303-1307
Szczeklik A, Gryglewski R J, Nizankowski R et al. Circulatory and anti-platelet effects of intravenous prostacyclin in healthy men. Pharmacol Res Commun 1978; 10:545-556
Thompson L F, Eltzschig H K, Ibla J C et al. Crucial role for ecto-5′-nucleotidase (C D73) in vascular leakage during hypoxia. J Exp Med 2004; 200:1395-1405
Xiang Y Z, Xia Y, Gao X M et al. Platelet activation, and antiplatelet targets and agents: current and novel strategies. Drugs 2008; 68:1647-1664
Xu J, Zhang X, Pelayo R et al. Extracellular histones are major mediators of death in sepsis. Nat Med 2009; 15:1318-1321
Yusuf S, Hawken S, Ounpuu S et al. Effect of potentially modifiable risk factors associated with myocardial infarction in 52 countries (the INTERHEART study): case-control study. Lancet 2004; 364:937-952
Zardi E M, Zardi D M, Cacciapaglia F et al. Endothelial dysfunction and activation as an expression of disease: role of prostacyclin analogs. Int Immunopharmacol 2005; 5:437-459
Zardi E M, Zardi D M, Dobrina A et al. Prostacyclin in sepsis: a systematic review. Prostaglandins Other Lipid Mediat 2007; 83:1-24

1.-31. (canceled)

32. A pharmaceutical composition comprising one or more compounds capable of modulating/preserving the endothelial integrity and one or more platelet inhibitors for use in the treatment and/or prevention of ischemic events in humans being at increased risk of developing an ischemic event.

33. The pharmaceutical composition of claim 32, wherein the ischemic event is an acute myocardial infarction and/or no-reflow phenomena and/or ischemia-reperfusion injury.

34. The pharmaceutical composition of claim 32, wherein the compound capable of modulating/preserving the endothelial integrity is selected from the group consisting of PGI2, PGX, nitrogen oxide, and prostacyclin or variants thereof.

35. The pharmaceutical composition of claim 32, wherein the compound capable of modulating/preserving the endothelial integrity is a prostacyclin variant selected from the group consisting of beraprost sodium, epoprostenol sodium, iloprost, iloprost in combination with bosentan, iloprost in combination with sildenafil citrate, treprostinil, pegylated treprostinil, treprostinil diethanolamine and treprostinil sodium, 2-{4-[(5,6-diphenylpyrazin-2-yl)(isopropyl)amino]butoxy}-N-(methylsulfonyl)acetamide, {4-[(5,6-diphenylpyrazin-2-yl)(isopropyl)amino]butoxy}acetic acid, 8-[1,4,5-triphenyl-1H-imidazol-2-yl-oxy]octanoic acid, isocarbacyclin, cicaprost, [4-[2-(1,1-Diphenylethylsulfanyl)-ethyl]-3,4-dihydro-2H-benzo[1,4]oxazin-8-yloxy]-acetic acid N-Methyl-d-glucamine, 7,8-dihydro-5-(2-(1-phenyl-1-pyrid-3-yl-methiminoxy)-ethyl)-a-naphthyloxyacetic acid, (5-(2-diphenylmethyl aminocarboxy)-ethyl)-a-naphthyloxyaceticacid, 2-[3-[2-(4,5-diphenyl-2-oxazolyl)ethyl]phenoxy]acetic acid, [3-[4-(4,5-diphenyl-2-oxazolyl)-5-oxazolyl]phenoxy]acetic acid, bosentan, 17[alpha], 20-dimethyl-[DELTA]6,6a-6a-carba PGI1, 15-deoxy-16[alpha]-hydroxy-16[beta], 20-dimethyl-[DELTA]6,6a-6a-carba PGI1 and pentoxifylline (1-{5-oxohexyl}-3,7-dimethylxanthine) and combinations thereof.

36. The pharmaceutical composition of claim 32, wherein the platelet inhibitor is capable of inhibiting the GPIIb/IIIa receptor.

37. The pharmaceutical composition of claim 32, wherein the platelet inhibitor is selected from the group consisting of eptifibatide, tirofiban, abciximab, orbofiban, xemilofiban, lamifiban, XJ757, DUP728, XR299 and combinations thereof.

38. The pharmaceutical composition of claim 32, wherein the compound capable of modulating/preserving the endothelial integrity is selected from the group consisting of PGI2, PGX, nitrogen oxide, and prostacyclin or variants thereof, a platelet inhibitor capable of inhibiting the GPIIb/IIIa receptor and combinations thereof.

39. The pharmaceutical composition of claim 32, wherein the compound capable of modulating/preserving the endothelial integrity is PGI2 (flolan) or prostacyclin (Epoprostenol) and the platelet inhibitor capable of inhibiting the GPIIb/IIIa receptor is eptifibatide (integrilin).

40. The pharmaceutical composition of claim 32, wherein the compound capable of modulating/preserving the endothelial integrity is selected from the group consisting of PGI2, PGX, nitrogen oxide, and prostacyclin or variants thereof, a platelet inhibitor capable of inhibiting platelet ADP receptor P2Y12 and combinations thereof.

41. The pharmaceutical composition of claim 32, wherein the platelet inhibitor is capable of inhibiting platelet ADP receptor P2Y12 is selected from the group consisting of Ticagrelor, Clopidogrel (Plavix), Prasugrel, cangrelor, Ticlopidine (Ticlid) and combinations thereof.

42. The pharmaceutical composition of claim 32, wherein the compound capable of modulating/preserving the endothelial integrity is selected from the group consisting of PGI2, PGX, nitrogen oxide, CD39, CD73, prostacyclin, beraprost sodium, epoprostenol sodium, iloprost, iloprost in combination with bosentan, iloprost in combination with one or more of sildenafil citrate, treprostinil, pegylated treprostinil, treprostinil diethanolamine and treprostinil sodium, 2-{4-[(5,6-diphenylpyrazin-2-yl)(isopropyl)amino]butoxy}-N-(methylsulfonyl)acetamide, {4-[(5,6-diphenylpyrazin-2-yl)(isopropyl)amino]butoxy}acetic acid, 8-[1,4,5-triphenyl-1H-imidazol-2-yl-oxy]octanoic acid, isocarbacyclin, cicaprost, [4-[2-(1,1-Diphenylethylsulfanyl)-ethyl]-3,4-dihydro-2H-benzo[1,4]oxazin-8-yloxy]-acetic acid N-Methyl-d-glucamine, 7,8-dihydro-5-(2-(1-phenyl-1-pyrid-3-yl-methiminoxy)-ethyl)-a-naphthyloxyacetic acid, (5-(2-diphenylmethyl aminocarboxy)-ethyl)-a-naphthyloxyaceticacid, 2-[3-[2-(4,5-diphenyl-2-oxazolyl)ethyl]phenoxy]acetic acid, [3-[4-(4,5-diphenyl-2-oxazolyl)-5-oxazolyl]phenoxy]acetic acid, bosentan, 17[alpha], 20-dimethyl-[DELTA]6,6a-6a-carba PGI1, 15-deoxy-16[alpha]-hydroxy-16[beta], 20-dimethyl-[DELTA]6,6a-6a-carba PGI1, pentoxifylline (1-{5-oxohexyl}-3,7-dimethylxanthine), and combinations thereof and a platelet inhibitor capable of inhibiting the GPIIb/IIIa receptor.

43. The pharmaceutical composition of claim 32, wherein the compound capable of modulating/preserving the endothelial integrity is selected from the group consisting of PGI2, PGX, nitrogen oxide, CD39, CD73, prostacyclin, beraprost sodium, epoprostenol sodium, iloprost, iloprost in combination with bosentan, iloprost in combination with sildenafil citrate, treprostinil, pegylated treprostinil, treprostinil diethanolamine and treprostinil sodium, 2-{4-[(5,6-diphenylpyrazin-2-yl)(isopropyl)amino]butoxy}-N-(methylsulfonyl)acetamide, {4-[(5,6-diphenylpyrazin-2-yl)(isopropyl)amino]butoxy}acetic acid, 8-[1,4,5-triphenyl-1H-imidazol-2-yl-oxy]octanoic acid, isocarbacyclin, cicaprost, [4-[2-(1,1-Diphenylethylsulfanyl)-ethyl]-3,4-dihydro-2H-benzo[1,4]oxazin-8-yloxy]-acetic acid N-Methyl-d-glucamine, 7,8-dihydro-5-(2-(1-phenyl-1-pyrid-3-yl-methiminoxy)-ethyl)-a-naphthyloxyacetic acid, (5-(2-diphenylmethyl aminocarboxy)-ethyl)-a-naphthyloxyaceticacid, 2-[3-[2-(4,5-diphenyl-2-oxazolyl)ethyl]phenoxy]acetic acid, [3-[4-(4,5-diphenyl-2-oxazolyl)-5-oxazolyl]phenoxy]acetic acid, bosentan, 17[alpha], 20-dimethyl-[DELTA]6,6a-6a-carba PGI1, 15-deoxy-16[alpha]-hydroxy-16[beta], 20-dimethyl-[DELTA]6,6a-6a-carba PGI1, pentoxifylline (1-{5-oxohexyl}-3,7-dimethylxanthine) and combinations thereof, and a platelet inhibitor capable of inhibiting platelet ADP receptor P2Y12, and combinations thereof.

44. The pharmaceutical composition of claim 32, wherein the compound capable of modulating/preserving the endothelial integrity is selected from the group consisting of PGI2, PGX, nitrogen oxide, CD39, CD73 and prostacyclin, beraprost sodium, epoprostenol sodium, iloprost, iloprost in combination with bosentan, iloprost in combination with sildenafil citrate, treprostinil, pegylated treprostinil, treprostinil diethanolamine and treprostinil sodium, 2-{4-[(5,6-diphenylpyrazin-2-yl)(isopropyl)amino]butoxy}-N-(methylsulfonyl)acetamide, {4-[(5,6-diphenylpyrazin-2-yl)(isopropyl)amino]butoxy}acetic acid, 8-[1,4,5-triphenyl-1H-imidazol-2-yl-oxy]octanoic acid, isocarbacyclin, cicaprost, [4-[2-(1,1-Diphenylethylsulfanyl)-ethyl]-3,4-dihydro-2H-benzo[1,4]oxazin-8-yloxy]-acetic acid N-Methyl-d-glucamine, 7,8-dihydro-5-(2-(1-phenyl-1-pyrid-3-yl-methiminoxy)-ethyl)-a-naphthyloxyacetic acid, (5-(2-diphenylmethyl aminocarboxy)-ethyl)-a-naphthyloxyaceticacid, 2-[3-[2-(4,5-diphenyl-2-oxazolyl)ethyl]phenoxy]acetic acid, [3-[4-(4,5-diphenyl-2-oxazolyl)-5-oxazolyl]phenoxy]acetic acid, bosentan, 17[alpha], 20-dimethyl-[DELTA]6,6a-6a-carba PGI1, 15-deoxy-16[alpha]-hydroxy-16[beta], 20-dimethyl-[DELTA]6,6a-6a-carba PGI1, pentoxifylline (1-{5-oxohexyl}-3,7-dimethylxanthine), and combinations thereof, and the platelet inhibitor aspirin.

45. The pharmaceutical composition of claim 32 further comprising one or more parenteral anticoagulants.

46. The pharmaceutical composition of claim 32 further comprising one or more compounds selected from the group consisting of Verapamil, aspirin, Sodium nitroprusside, Nitroglycerin, Beta-blockers, Epinephrine, norepinephrine, dopamine, dobutamine and combinations thereof.

47. The pharmaceutical composition of claim 32 wherein said one or more compounds capable of modulating/preserving the endothelial integrity is administered before, during and/or after a surgical procedure.

48. The pharmaceutical composition of claim 47 wherein the surgical procedure is Percutaneous Coronary Intervention (PCI), coronary angiography, Coronary artery bypass surgery (CABG) or thrombolytic therapy.

49. A method of treating or preventing ischemic events in humans being at increased risk of developing an ischemic event comprising administering one or more compounds capable of modulating/preserving the endothelial integrity and one or more platelet inhibitors.

50. The method of claim 49 wherein said human patient has acute coronary syndrome or purpura fulminans or is at risk of developing purpura fulminans or said patient has frostbite or is at risk of developing frostbite, or said patient has a wound, sore or ulcer or has melidioisis.

51. The method of claim 49 wherein the treatment and/or prevention is of the ischemic and/or ischemic reperfusion injuries that are associated with the presence of or removal of pulmonary emboli, intra cerebral venous and/or arterial thrombi and/or emboli, and gastrointestinal thromboses and/or emboli.

52. A method of treating or preventing cardiovascular ischemia comprising administering one or more compounds capable of modulating/preserving the endothelial integrity and one or more platelet inhibitors.

53. The method of claim 49 wherein said one or more compounds capable of modulating/preserving the endothelial integrity and one or more platelet inhibitor is administered before, during and/or after a surgical procedure.

54. The method of claim 49 further comprising administering one or more platelet inhibitors.

55. The method of claim 49 further comprising administering one or more Parenteral anticoagulants.

56. The method of claim 49 further comprising administering one or more compounds selected from the group consisting of Verapamil, Adenosine, Sodium nitroprusside, Nitroglycerin, Beta-blockers, Pressor drugs/rescue drugs, Epinephrine, norepinephrine, dopamine and dobutamine.

57. Use of one or more compounds capable of modulating/preserving the endothelial integrity and one or more platelet inhibitors in the manufacture of a medicament for the treatment or prevention of ischemic events in humans being at increased risk of developing an ischemic event.
